(19) United States
(12) Patent Application Publication
(10) Pub. No.: US 2026/0321390 A1
(43) Pub. Date: Oct. 15, 2026

(54) METHODS AND SYSTEMS FOR AUTOMATIC NUTRIMENT TRACKING

(71) Applicant: HealthifyMe Inc., Bangalore (IN)

(72) Inventors: Tushar VASHISHT, Bengaluru (IN); Abhijit KHASNIS, Bengaluru (IN); Aakar SHARMA, Bengaluru (IN); Jaswanth DEVARINTI, Bengaluru (IN); Jaishree GARG, Bengaluru (IN)

(21) Appl. No.: 19/274,195

(22) Filed: Jul. 18, 2025

(30) Foreign Application Priority Data
Apr. 9, 2025 (IN) 202541034822

(51) Int. Cl. G16H 20/60; G06F 21/62; G06V 10/764; G06V 10/82; G06V 20/30; G06V 20/68

(52) U.S. Cl. CPC G16H 20/60; G06F 21/6245; G06V 10/764; G06V 10/82; G06V 20/30; G06V 20/68; G06V 2201/03

(57) ABSTRACT

The systems, the methods, the computer-readable media, and the techniques disclosed herein may comprise: (A) obtaining permission from a user to access a camera roll comprising a plurality of images that span at least one week, wherein the camera roll is accessible to a mobile device; (B) obtaining a prediction, from a first machine learning model, comprising a plurality of nutriment parameters corresponding to each nutriment of each image of the plurality of images; and (C) presenting an insight to the user on the mobile device, wherein the insight is based at least in part on the plurality of nutriment parameters.

### CROSS REFERENCE

This application claims priority to Indian Patent Application No. 202541034822, filed Apr. 9, 2025, which is incorporated herein by reference in its entirety.

### BACKGROUND

A healthy diet may contain fruits, vegetables, meats, grains, oils, supplements, etc., with, for example, little to no processed foods or sweetened beverages. Various nutrition guides are published by medical and governmental institutions to educate individuals on what they should be eating to be healthy, while advertising may drive preferences towards unhealthy foods. To help offset the drive towards unhealthy foods from advertising, convenience factors, sugar addictions, etc. consumers should be informed, motivated and empowered to choose healthy diets. Some consumers will track their food purchases or food intake to try to make informed decisions for their dietary behavior.

### SUMMARY

Systems, methods, computer-readable media, and techniques disclosed herein

In an aspect, the present disclosure provides a computer-implemented method, comprising, obtaining permission from a user to access a camera roll comprising a plurality of images that span at least one week, wherein the camera roll is accessible to a mobile device, obtaining a prediction, from a first machine learning model, comprising a plurality of nutriment parameters corresponding to each nutriment of each image of the plurality of images, and presenting an insight to the user on the mobile device, wherein the insight is based at least in part on the plurality of nutriment parameters.

In some embodiments, the plurality of images span at least a month. In some embodiments, the plurality of images span at least one year. In some embodiments, the mobile device comprises one or more of a smartphone, a tablet, a smartwatch, smart glasses, other smart or IoT devices such as refrigerators, kitchen appliances, scales, utensils etc., or an augmented reality/virtual reality device. In some embodiments, a camera of the mobile device captured at least a first subset of the plurality of images. In some embodiments, a different device from the mobile device captured at least a second subset of the plurality of images. In some cases, the camera roll operates within a first software application of the mobile device and subsequent operations of the method are performed within a second software application of the mobile device. In some embodiments, the camera roll comprises one or more images captured on the mobile device. In some embodiments, the camera roll comprises one or more images posted to a social media account accessible by the mobile device. In some embodiments, the the first machine learning model comprises a large language model. In some embodiments, the plurality of nutriment parameters comprise two or more of, a type of nutriment, a quantity of nutriment, a size of nutriment, calories in nutriment, protein in nutriment, fats in nutriment, carbs in nutriment, or fiber in nutriment. In some embodiments, the insight comprises one or more of, a timeline of nutriment consumption, a trend or recommendation in consumption of one or more ingredients, a trend or recommendation in cooking preparation of one or more nutriments, a trend or recommendation in quantity of nutriment consumption, or a recommendation to align consumption with one or more goals of the user. In some embodiments, the computer-implemented method further comprises, obtaining an indication that the user suffers from at least one medical condition, and wherein the insight comprises one or both of recommending a nutriment for consumption based at least in part on the at least one medical condition, or recommending a nutriment for avoidance based at least in part on the at least one medical condition. In some embodiments, the computer-implemented method further comprises, identifying the plurality of images from the camera roll. In some embodiments, the plurality of images are identified from the camera roll using a second machine learning model configured to identify whether or not an image comprises a nutriment. In some embodiments, the computer-implemented method further comprises, transmitting the plurality of images to a computing device external to the mobile device, wherein the computing device comprises the first machine learning model. In some embodiments, the computer-implemented method further comprises, capturing an additional image of an additional nutriment in response to input from the user, obtaining an updated prediction, from the first machine learning model, comprising an additional plurality of nutriment parameters corresponding to the additional nutriment of the additional image, and presenting an updated insight to the user on the mobile device, wherein the updated insight is based at least in part on the additional plurality of nutriment parameters. In some embodiments, the computer-implemented method further comprises, presenting the plurality of nutriment parameters to the user, obtaining feedback from the user, and revising the plurality of nutriment parameters based at least in part on the feedback. In some embodiments, the nutriment comprises one or more of food, beverage, medication, supplement, vitamin, inhalant, or injection.

In another aspect, the present disclosure provides a computer implemented method, comprising, obtaining a plurality of images, using a first machine learning model, identifying a subset of the plurality of images that comprise a nutriment, providing a subset of the images as an input to a second machine learning model, obtaining a prediction, from the second machine learning model, comprising a plurality of nutriment parameters corresponding to each nutriment of each image of the plurality of images, and presenting an insight to the user on the mobile device, wherein the insight is based at least in part on the plurality of nutriment parameters.

In some embodiments, the plurality of images span at least one week. In some embodiments, the plurality of images span at least one month. In some embodiments, the plurality of images span at least one year. In some embodiments, the first machine learning model is performed using a mobile device that comprises one or more of a smartphone, a tablet, a smartwatch, smart glasses, other smart or IoT devices such as refrigerators, kitchen appliances, scales, utensils, etc., or an augmented reality/virtual reality device. In some embodiments, the camera of the mobile device captured at least a first subset of first plurality of images. In some embodiments, a different device from the mobile device captures at least a second subset of the plurality of images. In some embodiments, the plurality of images are obtained from a camera roll that operates within a first software application of the mobile device, and the subsequent operations of the computer-implemented method are performed within a second software application of the mobile device. In some embodiments, the first machine learning model comprises a neural network and the second machine learning model comprises a large language model. In some embodiments, the plurality of nutriment parameters comprises one or more of, a timeline of nutriment consumption, a trend or recommendation in consumption of one or more ingredients, a trend or recommendation in cooking preparation of one or more nutriments, a trend or recommendation in quantity of nutriment consumption, or a recommendation to align consumption with one or more goals of the user. In some embodiments, the computer-implemented method further comprises obtaining an indication that the user suffers from at least one medical condition, and wherein the insight comprises one or both of recommending a nutriment for consumption based at least in part on the at least one medical condition or recommending a nutriment for avoidance based at least in part on the at least one medical condition. In some embodiments, providing the subset of the plurality of images as input to a second machine learning model comprises transmitting the subset of the plurality of images to a computing device comprising the second machine learning model. In some embodiments, the computer-implemented method further comprises, capturing an additional image of an additional nutriment in response to input from the user, obtaining an updated prediction from the second machine learning model, comprising an additional plurality of nutriment parameters corresponding to the additional nutriment of the additional image, and presenting an updated insight to the user on the mobile device wherein the updated insight is based at least in part on the additional plurality of nutriment parameters. In some embodiments, the computer implemented method further comprises, presenting the plurality of nutriment parameters to the user, obtaining feedback from the user, and revising the plurality of nutriment parameters based at least in part on the feedback. In some embodiments, the nutriment comprises one or more of, food, beverage, medication, supplement, vitamin, inhalant, or injection.

In another aspect, the present disclosure provides a mobile computing device, comprising, one or more processors, and one or more memories storing computer-executable instructions that, when executed, cause the one or more processors to perform one or more aspects of the computer-implemented method described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. In some embodiments, the novel features of the present disclosure are set forth with particularity in the appended claims. In some embodiments, a better understanding of the features and advantages of the present disclosure will be obtained by reference to the following detailed description that sets forth illustrative embodiments, in which the principles of the disclosure are utilized, and the accompanying drawings of which:

FIG. 1 shows an example of a system diagram for a user device interacting with a server;

FIG. 2A shows an example of a flowchart of data for providing guidance and insights to a user;

FIG. 2B shows an example of a flowchart of a user experience for providing guidance and insights to a user;

FIGS. 3A-3F show a examples of user interfaces for analyzing a user's food;

FIGS. 4A and 4B show non-limiting examples of computer-implemented methods, in accordance with some embodiments.

FIG. 5 shows an example of a computer system configured to perform analysis of a user's food.

### DETAILED DESCRIPTION

While various embodiments have been shown and disclosed herein, it will be obvious to those skilled in the art that such embodiments are provided by way of example only. Numerous variations, changes, and substitutions may occur to those skilled in the art without departing from the systems, the methods, the computer-readable media, and the techniques provided herein.

In some cases, the systems, the methods, the computer-readable media, and the techniques disclosed herein provide for automatically tracking nutriment (e.g., food, vitamins, supplements, oil, etc.) intake via images. In some cases, the systems, the methods, the computer-readable media, and the techniques disclosed herein may leverage machine learning advances (e.g., computer vision, classifiers, etc.) to provide a seamless and user-friendly experience.

In some cases, the images may be captured by a user via the user's smartphone. In this example, the images may be analyzed by an application on the smartphone. The application may be function across IOS, Android, and other platforms, providing, for example, consistent (or near consistent) user experiences across platforms.

In some cases, nutriment tracking may be performed in the background of a user device (e.g., smartphone, tablet, personal computer, smartwatch, etc.). For example, users may not need to open an application configured to track nutriments in order for the application to track the nutriments. This may, for example, help to improve continuous, consistent tracking, with minimal interruption to the user.

In some cases, users may have the ability to review and modify tracked data corresponding to nutriment intake. For example, users may view tracked meals (e.g., meals tracked automatically in the background of the user device) and may correct any inaccuracies, helping to improve accuracy and usefulness of the tracking.

In some cases, tracked nutriment data may be used to generate personalized insights and guidance for users. For example, the personalized insights and guidance may include one or more of nutrition advice, exercise recommendation, or other wellness tips based at least in part on the tracked nutriment data (e.g., logged meals).

In some cases, the systems, the methods, the computer-readable media, and the techniques disclosed herein may generate a timeline of nutriment logs based at least in part on past nutriment photos from a user device (e.g., a camera roll). This may enable a user to visually revisit past nutriments consumed and highlight eating patterns. For example, the systems, the methods, the computer-readable media, and the techniques disclosed herein may provide feedback to the user, such as, “You tend to eat more carbs on the weekend.” Accordingly, through accessing a user's camera roll, the systems, the methods, the computer-readable media, and the techniques disclosed herein may provide insights on a user's eating habit at the outset of using the application (without needing to wait days, weeks, months, etc. to assemble enough data on nutriment consumption).

In some cases, the systems, the methods, the computer-readable media, and the techniques disclosed herein may integrate with wearable technology, such as smartwatches, smart glasses, medical devices, augmented reality/virtual reality (AR/VR) headsets, etc. For example, integration may enable hands-free food tracking. Users could capture and log meals through AR/VR interfaces, enhancing convenience and accessibility. In some cases, this integration with wearables may enable real-time portion estimation, ingredient identification, and context-aware meal suggestions during dining experiences.

In some cases, tracking nutriments includes tracking foods that a human (or animal) may consume. Some examples of foods that may be tracked by the systems, the methods, the computer-readable media, and the techniques disclosed herein may include one or more of: acai berries, acerola cherries, adzuki beans, almonds, aloo gobi, aloo matar, aloo tikki, amaranth, Amritsari fish, anchovies, anise, appam, apples, apricots, artichokes, arugula, asparagus, avocados, baati, badam kheer, bajji, bajra roti, baklava, bami goreng, banana chips, bananas, barley, basil, bass, bay leaves, BBQ sauce, bebinca, beef, beef rendang, beef vindaloo, beets, bell peppers, berries, bhakri, bhatura, bhelpuri, biscuits, bison, blackberries, black-eyed peas, blue cheese, blueberries, bok choy, bologna, bonito, boysenberries, Brazil nuts, bread, broccoli, brussels sprouts, buckwheat, bulgur, burdock root, butter, buttermilk, cabbage, cacao, cantaloupe, capers, cardamom, carrots, cashews, cauliflower, cayenne pepper, celery, chana masala, chappathi, chicken, chicken curry, chicken tikka masala, chikki, chives, chocolate, chole bhature, cilantro, cinnamon, clams, cloves, coconut, cod, coffee, coorgi coffee, collard greens, cookies, corn, corned beef, cottage cheese, couscous, crab, cranberries, cream cheese, cucumbers, cumin, curry leaves, curd rice, dal, dal makhani, dahi vada, dairy, dates, dill, dosa, duck, edamame, eggplant, eggs, elderberries, elk, endive, fava beans, fennel, figs, fish, fish curry, flaxseeds, fruit, fudge, gajar ka halwa, garlic, gelato, ginger, goat cheese, gooseberries, grains, grapefruit, grapes, green beans, green peppers, guava, gulab jamun, halibut, ham, hazelnuts, honey, honeydew, horseradish, hot dogs, hummus, ice cream, idli, jalapenos, jalebi, jelly, jicama, kachori, kale, ketchup, kheer, khichdi, kidney beans, kiwi, kofta, kohlrabi, kumquats, lamb, lamb biryani, lassi, leeks, lemongrass, lemons, lentils, lettuce, lima beans, limes, lobster, lychee, macadamia nuts, mackerel, mahi mahi, malai kofta, mangos, maple syrup, marjoram, masala chai, masala dosa, mayonnaise, meatloaf, melons, milk, mint, molasses, monkfish, morel mushrooms, muesli, mulberries, mussels, mustard greens, mysore pak, naan, nectarines, noodles, nutmeg, oats, oils, okra, olive oil, olives, onions, oranges, oregano, oysters, pancakes, papad, papaya, paprika, parsley, parsnips, passion fruit, pasta, peda, peaches, peanut butter, peanuts, pears, peas, pecans, peppers, persimmons, phirni, pickles, pine nuts, pineapple, pinto beans, pistachios, pizza, plums, poha, pomegranates, popcorn, pork, potatoes, protein, prunes, pulao, pumpkin, puri, quince, quinoa, radishes, raisins, rasgulla, raspberries, red onions, red peppers, rhubarb, rice, rogan josh, rosemary, rutabaga, rye, saffron, sage, salmon, salsa, sardines, scallops, seaweed, sesame seeds, shallots, shrimp, snapper, soda, sour cream, soy, spaghetti, spinach, squash, strawberries, sunflower seeds, sweet potatoes, Swiss chard, swordfish, tacos, tandoori chicken, tangerines, tarragon, tea, thali, thyme, tofu, tomatoes, trout, tuna, turkey, turnips, upma, uttapam, vada, vada pav, vanilla, veal, vegetable biryani, vegetable pulao, venison, vegetables, vinegar, walnuts, water chestnuts, watercress, watermelon, wheat, wheat germ, whipped cream, white wine, whole grains, wild rice, wine, yogurt, zucchini, etc. In some cases, tracking nutriments includes tracking vitamins that a human (or animal) may consume. Some examples of vitamins that may be tracked by the systems, the methods, the computer-readable media, and the techniques disclosed herein may include one or more of: vitamin A, vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folate, folic acid), vitamin B12 (cobalamin), vitamin C (ascorbic acid), vitamin D (calciferol), vitamin E (tocopherol), vitamin K (phylloquinone, menadione), etc. In some cases, tracking nutriments includes tracking supplements that a human (or animal) may consume. Some examples of supplements that may be tracked by the systems, the methods, the computer-readable media, and the techniques disclosed herein may include one or more of: 5-HTP, acetyl-l-carnitine, acidophilus, African mango, aged garlic extract, aloe vera, alpha GPC, alpha lipoic acid, amino acid complex, amla, Andrographis, arnica, artichoke, ashwagandha, astaxanthin, astragalus, b vitamins, bacopa, bee pollen, beetroot, berberine, beta carotene, beta glucan, biotin, bitter melon, black cohosh, black currant, black seed oil, blueberry, Boswellia, brewer's yeast, bromelain, cacao, calcium, cat's claw, chamomile, chaste tree, chia seed, chlorella, chlorophyll, chondroitin, chromium, cinnamon, citrulline, CLA, coenzyme Q10, collagen, colostrum, copper, cordyceps, cranberry, creatine, curcumin, dandelion, DHEA, devil's claw, digestive enzymes, Dong Quai, echinacea, elderberry, eleuthero, enzymes, EPA/DHA, eucalyptus, evening primrose oil, eyebright, fennel, fenugreek, feverfew, fiber, fish oil, flaxseed, folate, folic acid, fo-ti, frankincense, GABA, garlic, ginger, ginkgo biloba, ginseng, glucosamine, glutathione, goji, goldenseal, gotu kola, grape seed extract, grapeseed oil, green coffee bean, green tea extract, guarana, gymnema, hawthorn, hops, horny goat weed, horse chestnut, hyaluronic acid, iron, kava, kelp, krill oil, l-arginine, l-carnitine, l-glutamine, l-lysine, l-theanine, l-tryptophan, l-tyrosine, lavender, lecithin, lemon balm, licorice, lutein, lycopene, maca, magnesium, marshmallow, melatonin, milk thistle, moringa, MSM, multivitamins, NAC, neem, nettle, niacin, noni, olive leaf, omega-3 fatty acids, oregano oil, pantothenic acid, passion flower, Pau D'Arco, peppermint, phosphatidylserine, potassium, prebiotics, probiotics, psyllium, pumpkin seed, pygeum, quercetin, raspberry ketones, red clover, red yeast rice, reishi, resveratrol, rhodiola rosea, riboflavin, rose hip, rosemary, royal jelly, rutin, saffron, same, saw palmetto, selenium, senna, spirulina, St. John's Wort, stevia, taurine, tea tree oil, thiamine, tribulus, triphala, turmeric, ubiquinol, valerian, vitamin A, vitamin B1, vitamin B12, vitamin B2, vitamin B3, vitamin B5, vitamin B6, vitamin B7, vitamin B9, vitamin C, vitamin D, vitamin E, vitamin K, wheatgrass, white willow, zinc. In some cases, tracking nutriments includes tracking medications that a human (or animal) may consume. Some examples of medications that may be tracked by the systems, the methods, the computer-readable media, and the techniques disclosed herein may include one or more of: acetaminophen, albuterol, alendronate, allopurinol, alprazolam, amiodarone, amitriptyline, amlodipine, amoxicillin, atenolol, atorvastatin, azithromycin, bisoprolol, carbamazepine, cefalexin, cefuroxime, citalopram, clarithromycin, clindamycin, clopidogrel, codeine, cyclobenzaprine, dabigatran, dexamethasone, diazepam, diclofenac, digoxin, diltiazem, doxycycline, enalapril, escitalopram, esomeprazole, estradiol, etoricoxib, ezetimibe, famotidine, fentanyl, fluconazole, fluoxetine, fluticasone, furosemide, gabapentin, gliclazide, glimepiride, hydrochlorothiazide, hydrocodone, ibuprofen, insulin, isosorbide mononitrate, levothyroxine, lisinopril, losartan, lovastatin, metformin, metoclopramide, metoprolol, metronidazole, mirtazapine, montelukast, naproxen, omeprazole, oxycodone, paracetamol, pantoprazole, penicillin, phenytoin, prednisone, propranolol, ranitidine, rosuvastatin, sertraline, simvastatin, tamsulosin, tramadol, trazodone, valsartan, venlafaxine, warfarin, zolpidem, etc. In some cases, tracking nutriments includes tracking beverages that a human (or animal) may consume. Some examples of beverages that may be tracked by the systems, the methods, the computer-readable media, and the techniques disclosed herein may include one or more of: absinthe, aam panna, acai juice, alcoholic beverages, almond milk, aloo bukhara sharbat, amaretto, apple cider, apple juice, apricot juice, aquavit, arak, barley water, beer, bel ka sharbat, black coffee, black tea, Bloody Mary, bourbon, brandy, buttermilk, cacao, cappuccino, caramel macchiato, chai, champagne, chardonnay, cherry juice, cider, coconut milk, coconut water, coffee, cola, cosmopolitan, cranberry juice, cream soda, daiquiri, Darjeeling tea, decaf coffee, decaf tea, elderflower cordial, espresso, Frappuccino, fruit punch, gin, ginger ale, ginger beer, grape juice, green tea, grenadine, grappa, herbal tea, hot chocolate, iced coffee, iced tea, Irish cream, Irish whiskey, jal jeera, juice, kombucha, lassi, lemonade, lime cordial, liqueur, malt liquor, mango juice, margarita, martini, matcha tea, mead, merlot, milk, mint julep, mocha, mojito, moscato, mulled wine, nimbu pani, oat milk, orange juice, ouzo, peach juice, pina colada, pineapple juice, pisco, port, protein shake, red wine, root beer, rum, sake, sangria, scotch, sherry, shikanji, smoothie, soda, soju, soymilk, spiced rum, sprite, stout, strawberry juice, sula wine, tea, tequila, thandai, tonic water, vodka, water, white Russian, white wine, whiskey, whisky, wine, xeres, yogurt drink, zinfandel. It is worth noting that while food, vitamins, supplements, medications, and beverages are all disclosed herein as examples of nutriments that may be tracked by the systems, the methods, the computer-readable media, and the techniques disclosed herein, this is not exhaustive; anything else that may be ingested by a human (or animal) for providing nourishment, sustenance, growth, energy, maintaining of life, etc. (e.g., inhalants, injections, etc.) may be tracked by the systems, the methods, the computer-readable media, and the techniques disclosed herein.

### Examples of Systems for Tracking Nutriments

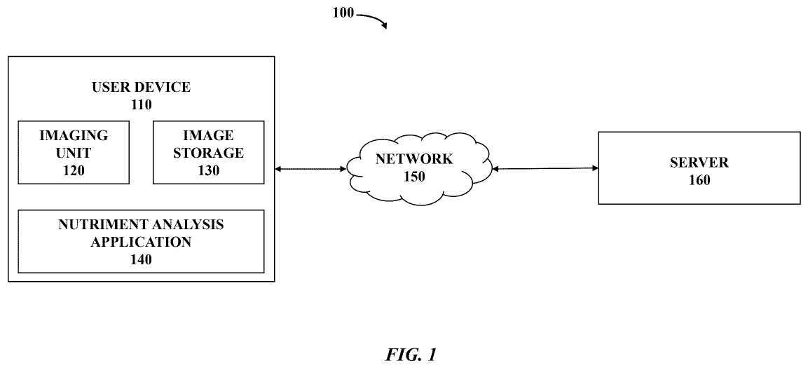

FIG. 1 shows an example of a system 100 for a user device 110 interacting with a server 160. As illustrated, the user device 110 may include an imaging unit 120, an image storage 130, and a nutriment analysis application 140. The user device 110 may communicate with the server 160 via a network 150.

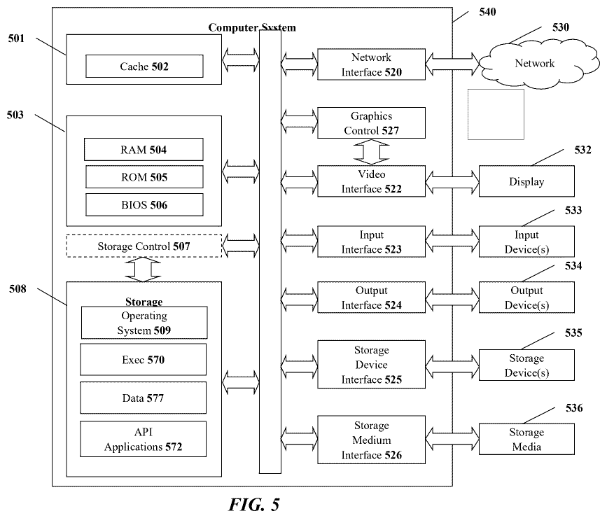

In some cases, the user device 110 may be one or more mobile devices. For example, the user device 110 may be one or more of: a smartphone, a tablet, a smartwatch, a laptop, smart glasses, medical devices, an augmented reality/virtual reality (AR/VR) headset, etc. Advantageously, in being a mobile device, a user of the user device 110 may be able to capture images of their food when the user is out and about as well as when at home. In some cases, the images of food may also comprise authorized tracking of social media posts. In some cases, the images of food may also comprise authorized tracking of scanned barcodes. In some cases, the images of food may also comprise nutrition labels. In some cases, the images of food may also comprise QR codes. In some cases, the images of food may also comprise other graphically coded identifiers. In some cases, the user device 110 may include a non-mobile device, such as a desktop computer. In some cases, the user device 110 may include a non-mobile device, such as smart kitchen devices. In some cases, the user device 110 may include smart kitchen devices such Internet of Things (IoT) and Artificial Intelligence (AI) enabled kitchen devices. In this case, the user device 110 may also include a mobile device that communicates with the non-mobile device. For example, the user may capture images of their food using the mobile device, which, may then transmit the images (or data corresponding thereto) to the non-mobile device (e.g., over the Internet, USB, Bluetooth, etc.). In another example, an IoT or AI enabled refrigerator or kitchen utensil may capture food information, which may then be transmitted to the mobile device. In some cases, the user device 110 may be the same as or similar to (in at least some components) the computer system 540 of FIG. 5.

In some cases, the imaging unit 120 may be configured to capture image data of nutriments (e.g., food, food ingredients or products, beverage, supplements, medication, vitamins, etc.). The image data may include, for example, still photographs, live photographs, video (with or without audio data), etc. The image data may correspond to one or more visible light, infrared light, ultraviolet light, etc. The imaging unit 120 may comprise hardware including one or more lenses, image sensors, apertures, shutters, viewfinders, focusing systems, displays, flashes, batteries, etc. In some cases, the imaging unit 120 may be permanently fixed to the user device 110, such as, a smartphone or tablet with an integrated camera. In other cases, the imaging unit 120 may be detachably fixed to the user device 110, such as, a smartphone or tablet physically (e.g., via USB) or remotely (e.g., via Bluetooth) connected to an external camera (e.g., a camera integrated into glasses of a user, a handheld camera, a camera integrated into an AR/VR headset, etc.). In some cases, the imaging unit 120 may be the same as or similar to (in at least some respects) the input devices 553 of the computing system 540 of FIG. 5.

In some cases, the image storage 130 may be configured to store image data captured by the imaging unit 120. The image storage 130 may store one or both of direct representations of the image data (e.g., actual photographs) or related data (e.g., computer vision analyses of the actual photographs, metadata of the actual photographs, etc.). In some cases, the image storage 130 may be the same as or similar to (in at least some respects) the storage 508 of FIG. 5.

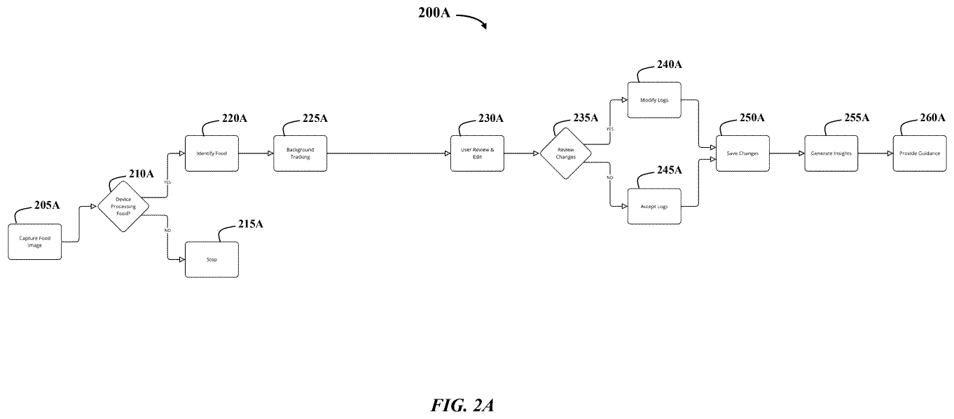

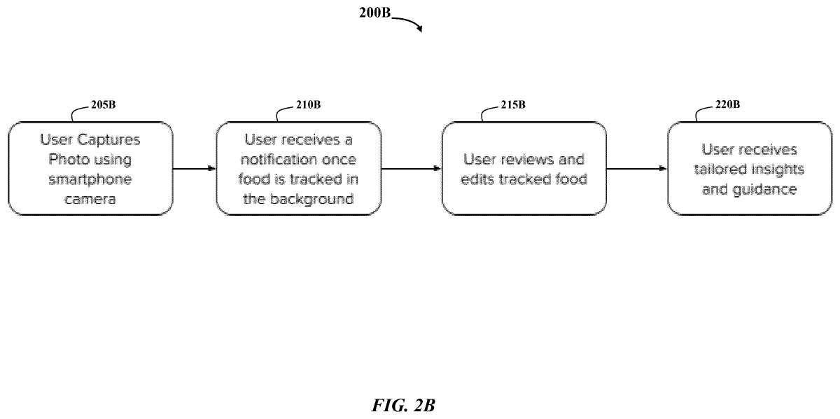

In some cases, the user device 110 may be configured to operate one or more applications, such as the nutriment analysis application 140. In some cases, the nutriment analysis application 140 may be downloadable to the user device 110, such as, from a digital store (e.g., the App Store from Apple, Google Play from Google, etc.). In other cases, the nutriment analysis application 140 may be a default or native application to the user device 110, not needing to be downloaded (and, for example, not removable) to the user device 110. In some cases, the nutriment analysis application 140 may be configured to perform at least some of the operations of the flowchart 200A or the flowchart 200B of FIG. 2A or 2B, respectively, or any other processes or methods disclosed herein. For example, the nutriment analysis application 140 may be configured to access images of nutriments, e.g., via one or both of the imaging unit 120 or the image storage 130. The nutriment analysis application 140 may perform one or more analyses (e.g., via machine learning models) on the images of the nutriments to track nutriments over time and generate insights or recommendations to a user. In some cases, such heuristic-based insights or recommendations to the user improve accuracy over traditional food tracking methods.

In some cases, the user device 110 may leverage the server 160 for computation, data storage, networking, etc. via connecting to the server 160 via the network 150 (which may be the same as or similar to the network 530 of FIG. 5). For example, the server 160 may help with storing image data captured by the imaging unit 120. In another example, the server 160 may help with performing computations (e.g., image analyses, insight generation, etc.) in order to improve computational efficiency via leveraging a greater processing power of the server 160 as compared to the user device 110. In some cases, the server 160 may gather data from many users (e.g., over time) in order to help develop, train, improve, refine, etc. machine learning models configured to perform at least some of the operations of the flowchart 200A or the flowchart 200B of FIG. 2A or 2B, respectively, or any other processes or methods disclosed herein. Advantageously, in leveraging data over many users (e.g., while encrypting or anonymizing the data), the systems, the methods, the computer-readable media, and the techniques disclosed herein may achieve greater accuracy in predictive ability. In some cases, the server 160 may be the same as or similar to (in at least some components) the computer system 540 of FIG. 5.

However, in some cases, the user device 110 may perform substantially all (or all) the operations of the flowchart 200A or the flowchart 200B of FIG. 2A or 2B, respectively, or any other processes or methods disclosed herein locally, without the use of the server 160. In these cases, advantageously, the user device 110 may operate without the use of the network 150 (e.g., without using cellular connectivity, Wi-Fi, the Internet, etc.). Further advantageously, operating substantially (or entirely locally) may present benefits of privacy, reduced network traffic, improved security and fraud prevention, etc.

### Examples of Flowcharts for Tracking Nutriments

FIG. 2A shows an example of a flowchart 200A of data for providing guidance and insights to a user. As illustrated, the flowchart 200A is from the perspective of data from the data's generation with capturing of a food (or other nutriment) image at block 205A and moving through various operations towards providing guidance at block 260A.

In some cases, the flowchart 200A may begin with capturing a food (or other nutriment) image at the block 205A. The image may be captured using an image capture device that may be the same as or similar to the imaging unit 120 of FIG. 1. The image may comprise a still photograph, a live photograph, a video etc. In some cases, the image may be captured in response to user input (e.g., a user triggering a shutter button). In other cases, the image may be captured automatically, such as, in response to detecting (e.g., via location sensing, audio sensing, visual sensing, behavioral prediction, etc.) a user is consuming or is about to consume a nutriment. In other cases, the image may be captured using authorized monitoring of social media accounts. In other cases, the image may be captured using authorized monitoring of wearable video and image capture devices such as smart glasses, Augmented Reality (AR) headsets, Mixed Reality (MR) headsets, and any other wearable video and image capture devices.

In some cases, the flowchart 200A may comprise identifying whether or not the image comprises an image of a nutriment at block 210A. For example, a “Food or No Food” (FNF) machine learning model may analyze the image captured at the block 205A to predict whether there is food (or another nutriments) included in the image. The FNF machine learning model may be a pre-trained mode performing locally on a device to quickly determine whether the image contains food. Advantageously, the FNF model improves computational efficiency and reduces network traffic by making a quick determination whether the image comprises food, thereby avoiding the need to perform a full image analysis, and increasing privacy protection of user data (e.g., with further computer vision models) in the event the image does not comprise food. Accordingly, if the image does not comprise food (or another nutriments), the flowchart 200A advances to a stop function at block 215A, ceasing the analysis (at least until a new image is captured at the block 205A).

In some cases, if the FNF model detects the image comprises food (or other nutriment), the flowchart continues to identify the food (or other nutriments) at block 220A. This identification may be performed using a machine learning model, such as a “Food Recognition Model” (FRM). The FRM, in some cases, may be a machine learning model that identifies nutriment items present in an image and estimates nutritional content of the nutriment using one or more comprehensive nutriment databases. For example, the FRM may be cloud-based. Advantageously, before expending the computational and network expense of uploading the image for cloud-based analysis using FRM, the image should already have been confirmed, via the FNF (e.g., locally on a user device), as comprising a nutriment.

In addition or in alternative to receiving and analyzing images in accordance with the blocks 205A-220A, in some cases, the flowchart 200A may include background tracking of food (or other nutriments). In some cases, this background food tracking means users do not need to provide input or open a tracking application (e.g., the nutriment analysis application 140) to log food. This enhances convenience and ensures continuous tracking without interruptions. For example, background food tracking at the block 225A may automatically activate food tracking when images are captured, even if the tracking application is not actively open. This may integrate with device-level permissions and camera and photo gallery or memory access to provide a seamless, low-intervention user experience. In another example, background tracking may include importing images from a user device's camera roll into the tracking application (e.g., either automatically, or in response to user request). In some cases, this background food at the block 225A may similarly perform a FNF model to identify whether there is food in the image analyzed in the background and may perform a FRM model to classify the food in the image.

In some cases, a user may be able to review and edit the food analyses performed in the blocks 205A-225A at block 230A. For example, the user may be able to review the outcome of the FNF model and may be able to change the determination by the FNF model. In another example, the user may be able to review the outcome of the FRM model and may be able to change the determination by the FRM model. Accordingly, the systems, the methods, the computer-readable media, and the techniques disclosed herein may provide, at the block 230A, a user feedback loop, allowing users to review and edit automatically logged entries, providing corrections or confirming accuracy. The flowchart shows at block 235A how changes proposed by a user or automatically may be reviewed, how logs may be modified at block 240A, how logs may be accepted at block 245A, and how changes may be saved at block 250A.

In some cases, once the logs are saved at the block 250A, the flowchart may include generating insights at block 255A. For example, the insights may be personalized to the user. These insights may drive comprehensive lifestyle feedback for the user and support tailored health recommendations. In some cases, personalization to the user may comprise user preference, user history, authorized location information, and other authorized metrics which aide in personalization to the user.

In some cases, once the logs are saved at the block 250A, the flowchart may include providing guidance at block 260A. In some cases, the guidance may be based at least in part on the insights generated at the block 255A. In one example, the insight/guidance may include oil usage, including estimating the quantity and type of oil used in preparation. In another example, the insight/guidance may include heat and cooking methods, including identifying if the food is grilled, fried, or steamed, and assess the impact on nutritional content. In another example, the insight/guidance may include quantity and quality, including accurately determining portion sizes and evaluate ingredient quality (e.g., freshness, organic vs. processed, etc.). In another example, the insight/guidance may include warning a user that a food they are about to consume may contain an allergen of the user. In another example, the insight/guidance may include a “snap rewind” functionality, including offering users a visual and analytical history of their eating habits over weeks, months, or even years, where, by analyzing trends in food choices, portion sizes, and cooking styles, users may receive insights into how their diet evolves over time, enabling better long-term health decisions. In another example, the insight/guidance may include an insight system for analyzing a user's food logs to identify patterns, highlight areas for improvement, and offer actionable suggestions (e.g., balance macronutrient intake, reduce unhealthy fats, increase fiber). In another example, the insight/guidance may include integrations with the user's fitness goals or medical conditions to provide tailored advice. In another example, the insight/guidance may include preventative insights tailored to a user's condition, such as suggesting low glycemic index foods based on logged meals for someone with diabetes, highlighting foods with high sodium content and recommending alternatives for someone with hypertension, automatically flagging gluten-containing foods for someone with Celiac disease, etc. Advantageously, the insights/guidance may monitor long-term trends to develop recommendations to help users mitigate health risks (e.g., overconsumption of sugar or fats). In some cases, the insights/guidance may be delivered via a user interface that enables users to interact with the feature, including viewing, editing, and confirming logged meals, as well as viewing recommendations and insights.

FIG. 2B shows an example of a flowchart 200B of a user experience for providing guidance and insights to a user. As illustrated, the flowchart 200B is from the perspective of a user using or interacting with nutriment tracking according to the systems, the methods, the computer-readable media, and the techniques disclosed herein.

In some cases, the flowchart 200B may begin with the user capturing an image at block 205B. The user may capture the image with a camera (which may be the same as or similar to the imaging unit 120 of FIG. 1). The camera may be integrated into another device, such as, a smartphone, a tablet, a computer, smart glasses, a smartwatch, an AR/VR headset, etc. Alternatively, the camera may be a standalone device, providing image data to another device.

In some cases, the flowchart 200B may continue with tracking food (or other nutriments) in the image. In some cases, the image may be captured by the user within a tracking application (e.g., the nutriment analysis application 140 of FIG. 1). In other cases, the image may be captured by the user outside the tracking application, and, instead, may be imported into the tracking application either automatically (in the background) or in response to the user selecting the images to be imported. At block 210B, the user may be notified that food (or other nutriments) have been tracked. For example, the tracking may be performed using one or both of the FNF or the FRM models.

In some cases, the flowchart 200B may continue with the user reviewing and editing tracked food at block 215B. This may include the user reviewing and editing whether or not there was food in an image (the classification from the FNF model) or reviewing and editing what type of food, the name of the food, and quantity of the food in the image (the classification from the FRM model).

In some cases, the flowchart 200B may continue with the user receiving tailored insights and guidance at block 220B. The insights and guidance of the block 220B may be the same as or similar to the insights and guidance disclosed herein with respect to the blocks 255A and 260A of FIG. 2A.

In some cases, one or more operations of one or both of the flowcharts 200A or 200B may be performed in any order. Further, at least one of the one or more operations disclosed above with respect to one or both of the flowcharts 200A or 200B may be repeated, e.g., iteratively.

### Examples of User Interfaces

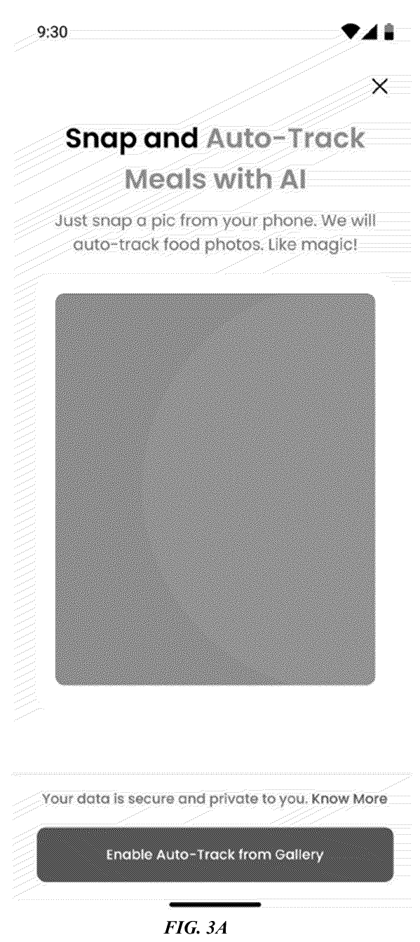

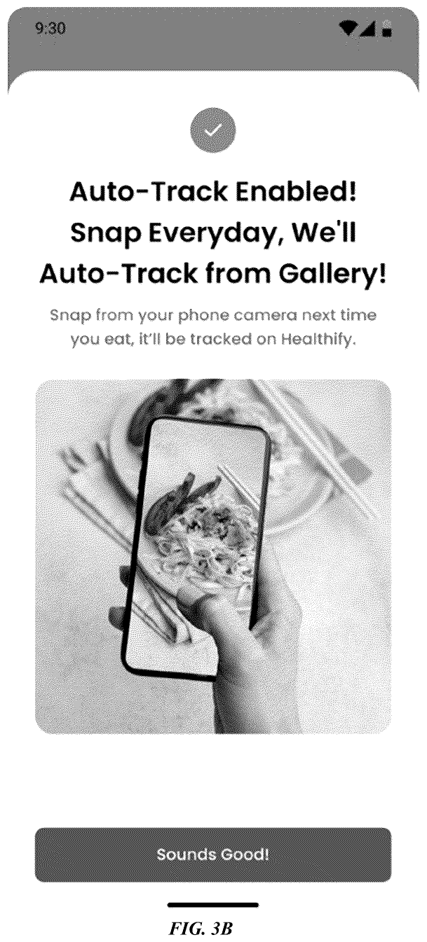

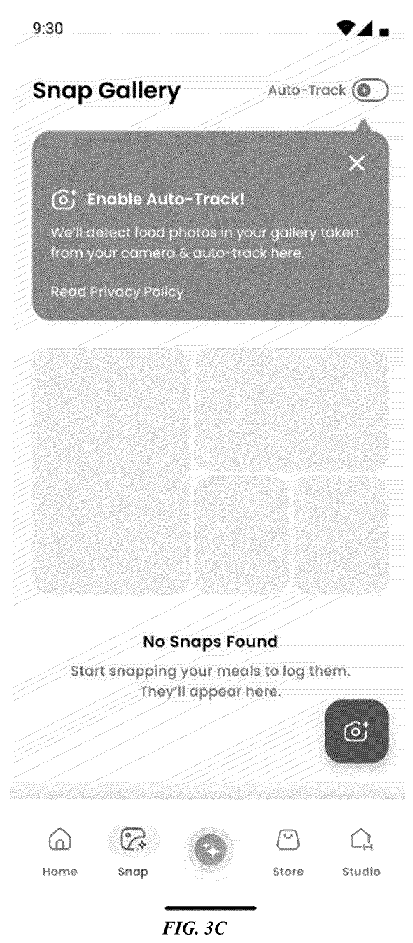

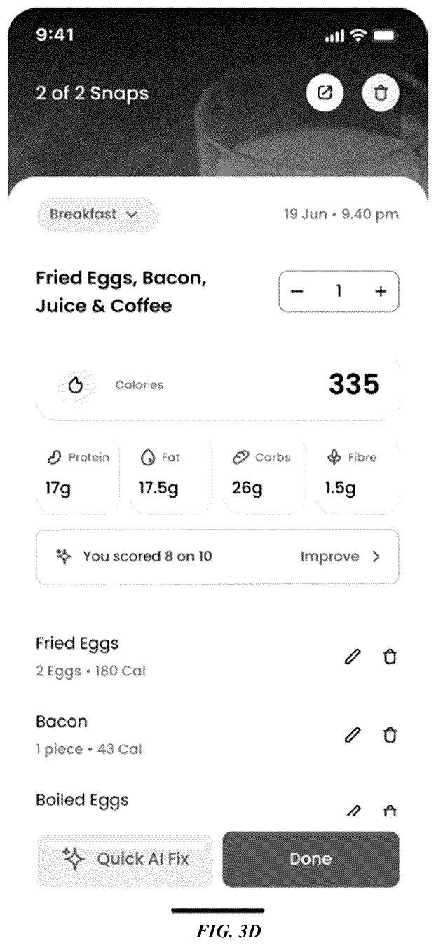

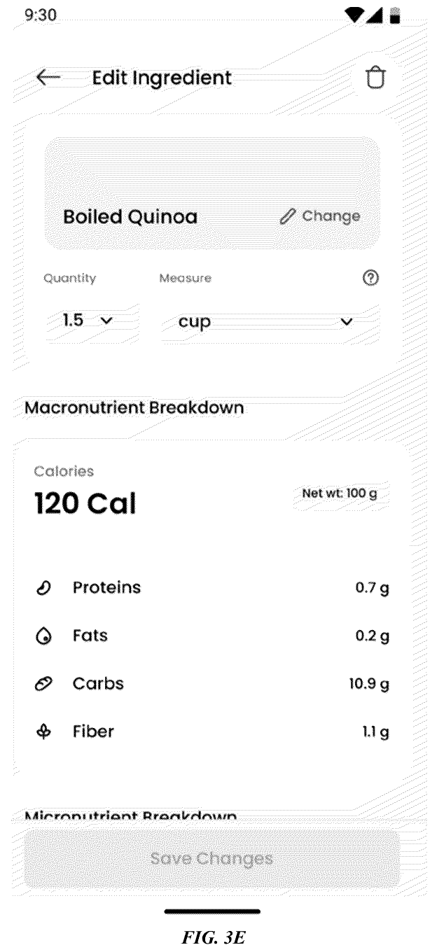

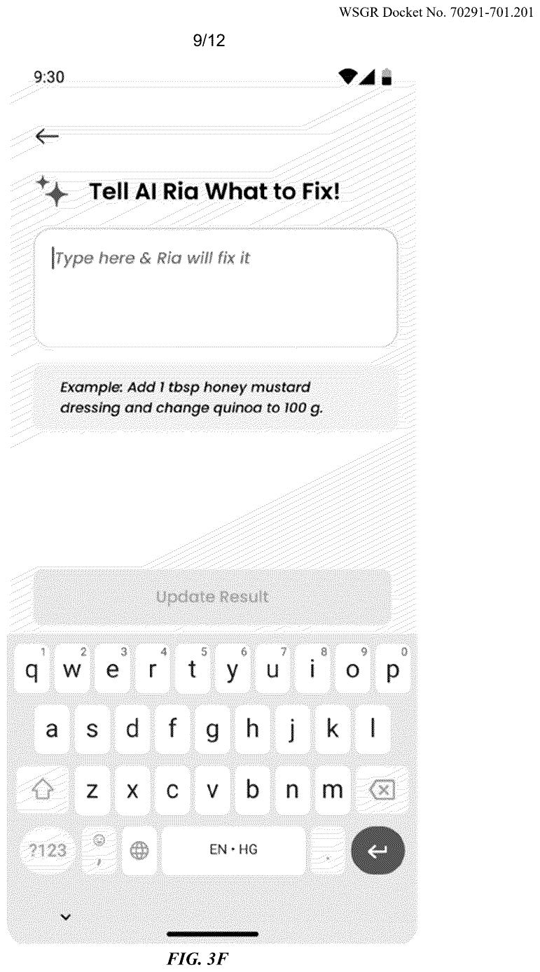

FIGS. 3A-3F show a examples of user interfaces 300A-300F for tracking and analyzing nutriment consumption by a user. The user interfaces 300A-300F may be configured to be presented on a user device, such as a user device that may be the same as or similar to the user device 110 of FIG. 1. For example, the user interfaces 300A-300F may be configured to be presented within the nutriment analysis application 140. The user interfaces 300A-300F may be configured to send or receive information (e.g., data) from the server 160. Further, the user interfaces 300A-300F may aid in performing operations of the flowcharts 200A or 200B of FIG. 2A or 2B, respectively.

The user interface 300A may present an interface for helping capture images (e.g., images of nutriments). For example, the user interface 300A may allow users to directly capture images of nutriments. Further, the user interface 300A may allow users to grant access permission to a camera roll (or other local storage or cloud storage on or associated with the user device) of a user's device. In this case, images from the camera roll may be automatically monitored, at some interval, or may be prompted automatically by the device or storage location when a new image is added, to identify when the camera roll adds (e.g., via a user taking a photograph) an image of a nutriment (e.g., food). For example, the interval may be one or more times a minute (e.g., about every second, every 5 seconds, every 20 seconds, etc.), about once an hour, about once every four hours, about twice a day, about once a day, about twice a week, about once a week, etc.

The user interface 300B may present confirmation of the user's selection and permission to automatically monitor a camera roll of a user device.

The user interface 300C may present additional opportunity for the user to enable or disable automatic tracking of images to identify nutriments. Further, the user interface 300C may display image previews of images. In one example, these images may include identified nutriments. In another example, these images may be images to be analyzed for nutriments. In another example, these images may serve as a preview of a larger group of images which will be analyzed for nutriments as a result of the user granting permission. Further, the user interface 300C, provides a functionality for the user to directly capture an image of nutriments in real-time.

The user interface 300D may present an opportunity for a user to review or edit one or more images. For example, as illustrated, the user is shown an indication that fried eggs, bacon, juice, and coffee were identified in one or more images on June 19^{th} at 9:40 PM. The user is shown the predicted number of calories, grams of protein, grams of fat, grams of carbs, grams of fiber. The user is also shown a breakdown of calories between each individual item of their meal. The user is also shown a Health Score that is based at least in part on one or more nutritional indexes related to the contents of the nutriments (e.g., nutrients). This Health Score may be further influenced by user input parameters including, but not limited to food sensitivities, physiological attributes such as height and weight, specific caloric or nutrient intake goals, and the like. The user interface 300D may include abilities to edit nutriment parameters, such as changing the type of nutriment (e.g., apple, banana, orange, etc.), the quantity of the nutriment, the size of the nutriment, the ingredients in the nutriment, the nutritional value (e.g., calories, proteins, fats, carbs, fiber, etc.) of the nutriment, etc. The user interface 300D may include a “Quick AI Fix” feature that enables providing semantic feedback on an identification of a nutriment.

The user interface 300E may present functionality for editing nutriments identified from an image. As illustrated, a user may edit nutriment parameters of a boiled quinoa identification, such as changing the type of nutriment, the quantity of the nutriment, the size of the nutriment, the ingredients in the nutriment, the nutritional value (e.g., calories, proteins, fats, carbs, fiber, etc.) of the nutriment, etc.

The user interface 300F may present functionality for providing semantic feedback on an identification of a nutriment, such as in response to a user selecting “Quick AI Fix” in the user interface 300D. As illustrated, a user may provide, as input to a machine learning model, feedback on nutriment parameters, such as changing the type of nutriment, the quantity of the nutriment, the size of the nutriment, the ingredients in the nutriment, the nutritional value (e.g., calories, proteins, fats, carbs, fiber, etc.) of the nutriment, etc. without having to enter these changes into a structured data format. For example, the user may provide instruction in sentence format with slang, misspellings, abbreviations, etc., which the machine learning model may be configured to standardize and interpret. In the illustrated example, the user may provide the instruction of “Add 1 tbsp honey mustard dressing and change quinoa to 100 g.” Here, the machine learning model will interpret “tbsp” to mean tablespoon, “100 g” to mean 100 grams.

### Examples of Personalization

In some cases, the systems, the methods, the computer-readable media, and the techniques disclosed herein may obtain information about a user to aid in generating personalized insights and improving accuracy in nutriment identification. For example, a user may provide their age range (or particular age), sex, height range (or particular height), weight range (or particular weight), level of physical activity, duration of physical activity, intensity of physical activity, daily exercise, weekly exercise, monthly exercise, yearly exercise, lifetime exercise, exercise trends, activity trends, exercise schedule, future exercise goals, future activity goals, ethnicity, ancestry, diet, allergies, food preferences, caloric intake goals, nutrient intake goals, medication history, medical history, employment, location of residency, health and wellness goals, etc.

In some cases, based at least in part on the obtained information about the user, the systems, the methods, the computer-readable media, and the techniques disclosed herein may generate a personalized nutriment program. In some cases, the nutriment program may include a nutriment plan, such as, indicating recommended nutriments that a user should include in their diet. The recommended nutriments may aid in achieving objectives of the user, such as alleviating symptoms a user suffers from, reaching a target weight, avoiding an allergen, improving metrics in a blood panel, etc. The personalized nutriment program may be displayed to the user using a graphical display.

In some cases, the personalized nutriment program recommends one or more foods to provide a recommended nutriment to the user. The one or more foods may be selected from a list of foods. In some cases, the list of foods may include foods on a menu, such as, a restaurant menu, a bar menu, etc. To obtain the menu, in one example, a photograph of the menu may be obtained (e.g., via a smartphone camera). In another example, a restaurant or bar name may be provided such that the menu may be obtained. In some cases, the list of foods may include foods in a store, such as, a convenience store, a grocery store, a market, etc. In this case, the list of foods may be obtained from a photograph (e.g., captured by a smartphone camera from inside the grocery store, the convenience store, the market, etc.) or from importing stock data (e.g., via integrations with the grocery store, the convenience store, the market, etc.). In some cases, the list of foods may include foods in a home (e.g., in a refrigerator, in a freezer, in a pantry, etc.). In this case, the list of foods may be obtained from a photograph (e.g., captured by a smartphone camera from inside the home) or from importing data from smart appliances (e.g., a smart refrigerator). Across these cases, the photographs may be, in some examples, photographs of the food item itself (e.g., leveraging computer vision to identify the food item. In other examples, the photographs may be of a written description of the food item (e.g., on packaging, on a label, on a menu, etc.). In other examples, the photographs may be of a code corresponding to the food item (e.g., a QR code, a barcode, a UPC code, etc.).

In some cases, the systems, the methods, the computer-readable media, and the techniques disclosed herein analyze users' food logs to identify patterns, highlight areas for improvement, and offer actionable suggestions (e.g., balance macronutrient intake, reduce unhealthy fats, increase fiber, etc.). In some cases, the systems, the methods, the computer-readable media, and the techniques disclosed herein may integrate with a user's fitness goals or medical conditions to provide tailored advice. This may include providing preventative insights tailored to a user's condition, such as suggesting low glycemic index foods based on logged meals for someone with diabetes, highlighting foods with high sodium content and recommending alternatives for someone with hypertension, automatically flagging gluten-containing foods for someone with Celiac disease, etc. In other cases, the methods, the computer-readable media, and the techniques disclosed herein may integrate with AI-generated predictive insights based on historical data trends.

### Examples of Methods for Tracking Nutriments

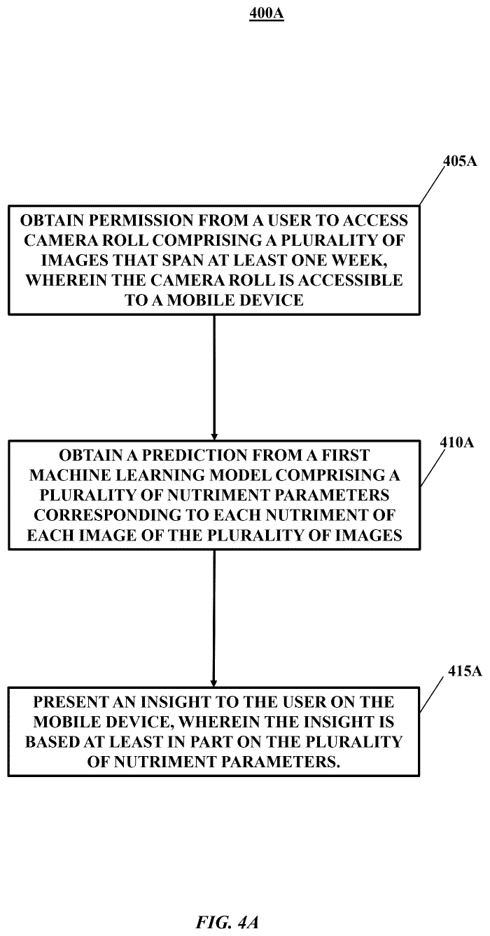

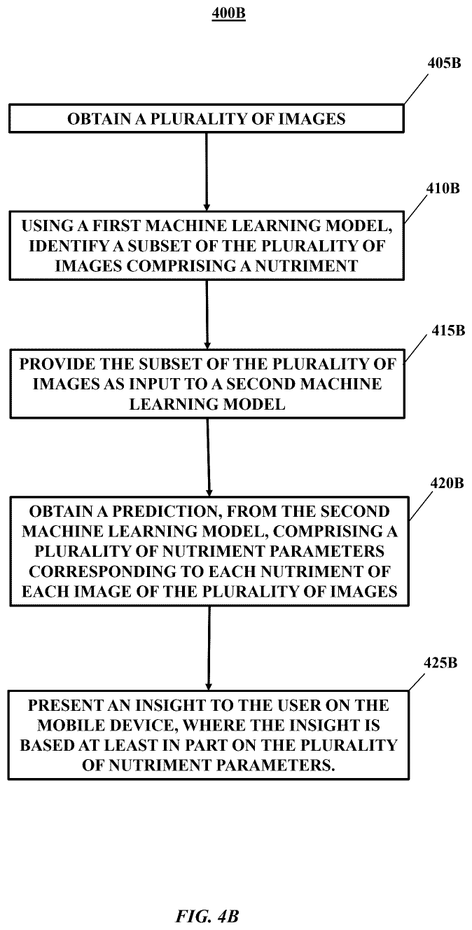

A non-limiting example of a computer-implemented method 400A is provided in FIG. 4A, in accordance with some embodiments. The computer-implemented method 400A comprises the operations of 405A, obtaining permission from a user to access the camera roll comprising a plurality of images that span at least one week, wherein the camera roll is accessible to a mobile device, 410A, obtaining a prediction from a first machine learning model comprising a plurality of nutriment parameters corresponding to each nutriment of each image of the plurality of images, and 415A, presenting an insight to the user on the mobile device, wherein the insight is based at least in part on the plurality of nutriment parameters.

The computer-implemented method 400A optionally further comprises obtaining an indication that the user suffers from at least one medical condition, and wherein the insight comprises one or both of recommending a nutriment for consumption based at least in part on the at least one medical condition or recommending a nutriment for avoidance based at least in part on the at least one medical condition. The computer-implemented method 400A optionally further comprises identifying the plurality of images from the camera roll. The computer-implemented method 400A optionally further comprises transmitting the plurality of images to a computing device external to the mobile device, wherein the computing device comprises the first machine learning model. The computer-implemented method 400A optionally further comprises capturing an additional image of an additional nutriment in response to input from the user; obtaining an updated prediction, from the first machine learning model, comprising an additional plurality of nutriment parameters corresponding to the additional nutriment of the additional image; and presenting an updated insight to the user on the mobile device, wherein the updated insight is based at least in part on the additional plurality of nutriment parameters. The computer-implemented method 400A optionally further comprises presenting the plurality of nutriment parameters to the user; obtaining feedback from the user; and revising the plurality of nutriment parameters based at least in part on the feedback.

A non-limiting example of a computer-implemented method 400B is provided in FIG. 4B, in accordance with some embodiments. The computer-implemented method 400B comprises the operations of 405A, obtaining a plurality of images, 410B, using a first machine learning model, identifying a subset of the plurality of images that comprise a nutriment, 415B, providing the subset of the plurality of images as input to a second machine learning model, 420B, obtaining a prediction, from the second machine learning model, comprising a plurality of nutriment parameters corresponding to each nutriment of each image of the plurality of images, and 425B, presenting an insight to the user on the mobile device, wherein the insight is based at least in part on the plurality of nutriment parameters.

The computer-implemented method 400B optionally further comprises obtaining an indication that the user suffers from at least one medical condition, and wherein the insight comprises one or both of recommending a nutriment for consumption based at least in part on the at least one medical condition or recommending a nutriment for avoidance based at least in part on the at least one medical condition. The computer-implemented method 400B optionally further comprises capturing an additional image of an additional nutriment in response to input from the user; obtaining an updated prediction, from the first machine learning model, comprising an additional plurality of nutriment parameters corresponding to the additional nutriment of the additional image; and presenting an updated insight to the user on the mobile device, wherein the updated insight is based at least in part on the additional plurality of nutriment parameters. The computer-implemented method 400B optionally further comprises presenting the plurality of nutriment parameters to the user; obtaining feedback from the user; and revising the plurality of nutriment parameters based at least in part on the feedback.

### Examples of Machine Learning Techniques

In some cases, the systems, the methods, the computer-readable media, and the techniques disclosed herein may leverage one or more machine learning techniques. For example, machine learning models may be used to help run the FNF model. In another example, machine learning models may be used to help run the FRM model. In another example, machine learning models may be used to help generate insights and recommendations for a user. In some cases, the systems, the methods, the computer-readable media, and the techniques disclosed herein may leverage neural networks, computer vision techniques, large language models, etc. to perform any of the methods or processes disclosed herein, such as with respect to the flowcharts 200A or 200B of FIG. 2A or 2B, respectively, or the methods and the systems 400A or 400B of FIG. 4A or 4B, respectively.

In some cases, machine learning (ML) may generally involve identifying and recognizing patterns in existing data in order to facilitate making predictions for subsequent data. ML may include a ML model (which may include, for example, a ML algorithm). Machine learning, whether analytical or statistical in nature, may provide deductive or abductive inference based on real or simulated data. The ML model may be a trained model. ML techniques may comprise one or more supervised, semi-supervised, self-supervised, or unsupervised ML techniques. For example, an ML model may be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, residual neural networks, physics-informed neural networks, long short-term memory, deep belief networks, deep Boltzmann machines, deep convolutional neural networks, deep recurrent neural networks, large language models, transformer models, vision transformers, or generative adversarial networks.

Training the ML model may include, in some cases, selecting one or more untrained data models to train using a training data set. The selected untrained data models may include any type of untrained ML models for supervised, semi-supervised, self-supervised, or unsupervised machine learning. The selected untrained data models may be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models may likewise be selected, such as limits on the ML model complexity or limits on the ML model refinement past a certain point. The ML model may be trained (e.g., via a computer system such as a server) using the training data set. In some cases, a first subset of the training data set may be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model.

In some cases, one or more aspects of the ML model may be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model may then be evaluated to determine whether performance is sufficient based upon the derived predictions. The sufficiency criteria applied to the ML model may vary depending upon the size of the training data set available for training, the performance of previous iterations of trained models, or user-specified performance requirements. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML may be stored for present or future use. The ML model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of ML models may be stored for generating predictions under different sets of input data conditions. In some cases, the ML model may be stored in a database (e.g., associated with a server)

### Computer Vision

The systems, the methods, the computer-readable media, and the techniques disclosed herein may implement one or more computer vision techniques. Computer vision is a field of artificial intelligence that uses computers to interpret and understand the visual world at least in part by processing one or more of images, videos, or even in some cases, audio. This involves transforming the input by disentangling symbolic information contained in the images, videos, or audio, to provide an output. This disentangled output may then be utilized as further inputs to further models, or as an input to a decision matrix or other process. In this way, computer vision may be used for object recognition, object classification, identification, detection, and other specialized tasks to name a few. In some instances, computer vision may use deep learning models (e.g., convolutional neural networks).

Computer vision may be utilized in the food detection process of the FNF machine learning model disclosed herein. Computer vision may be utilized to disentangle symbolic information contained in images and videos input to the FNF model. This disentangled information may comprise a food-likeliness score determined by one or more disentangled variables. The disentangled information of the FNF model may further comprise an input to one or more subsequent computer vision operations to update or further refine the food-likeliness score. The FNF model may comprise a regressive determination of a food-likeliness score for a given input or set of inputs. As an example, a component of the FNF machine learning model may comprise using computer vision to identify a food-likeliness score for portions of an image or video. As another example, a component of the FNF machine learning model may further comprise a plurality of subsequent computer vision operations directed to portions of an image or video possessing a high food-likeliness score. Advantageously, the output of the FNF model (e.g., through leveraging computer vision) may be a binary output of food or no food (or nutriment or no nutriment).

Computer vision may be utilized in the identification of nutriment items of the FRM machine learning model disclosed herein. Computer vision may be utilized to disentangle symbolic information contained in images and videos input to the FRM model. This disentangled information may comprise a matrix of nutriment-likeliness scores for a plurality of nutriments contained in one or more comprehensive nutriment databases. The disentangled information of the FRM model may further comprise an input to one or more subsequent vision operations to update or further refine the matrix of nutriment-likeliness scores. The FRM model may comprise a regressive determination of one or more nutriment likeliness scores for a given input or set of inputs. The FRM model may use stored historical data to update one or more nutriment likeliness scores for a given input or set of inputs. As an example, a component of the FRM machine learning algorithm may comprise using computer vision to identify one or more nutriment-likeliness scores for a plurality of nutriments, such as those present in one or more comprehensive nutriment databases. As another example, a component of the FRM machine learning algorithm may comprise using computer vision to identify one or more nutriment-likeliness scores for a plurality of nutriments, for specific portions of an image or video. As another example, a component of the FRM machine learning model may further comprise a plurality of subsequent computer vision operations directed to portions of an image or video possessing one or more high nutriment-likeliness scores.

Computer vision may be used to determine the amounts of nutriment in the FRM machine learning model disclosed herein. Computer vision may be used to disentangle symbolic information contained in images and videos input to the FRM model. This disentangled information may comprise a matrix of one or more relative size scores or one or more relative amount scores for disentangled members of the input. This disentangled information may comprise a matrix of relative size scores or relative amount scores for a plurality of nutriments contained in one or more comprehensive nutriment databases. This disentangled information may comprise a matrix of relative size scores or relative amount scores for a plurality of non-nutriment members of the image or video. The relative size scores or relative amount scores may be updated regressively. The FRM model may comprise a regressive determination of one or more relative size scores or one or more relative amount scores for a given input or set of inputs. The FRM model may use stored historical data to update one or more relative size scores or relative amount scores for a given input or set of inputs. As an example, a component of the FRM machine learning model may comprise using computer vision to identify and separate elements of an input or set of inputs. As an example, a component of the FRM machine learning model may further comprise weighting the separate elements of the input or set of inputs by relative size or amount. As an example, a component of the FRM machine learning model may further comprise identifying nutriment elements of the input or set of inputs, as disclosed herein. As an example, a component of the FRM machine learning model may further comprise identifying non-nutriment elements of the input or set of inputs. As an example, a component of the FRM machine learning model may further comprise using historical information about nutriment element sizes or amounts to update relative size scores or relative amount scores for an input or set of inputs. As an example, a component of the FRM machine learning model may further comprise using historical information about non-nutriment element sizes or amounts to update relative size scores or relative amount scores for an input or set of inputs. As an example, the FRM machine learning model may recognize a nutriment element, such as an apple, in the input or set of inputs, and use historical information about the size of an apple to update the relative size scores or relative amount scores for other elements contained in the input or set of inputs. As an example, the FRM machine learning model may recognize a non-nutriment item, such as a fork, in the input or set of inputs, and use historical information about the size of a fork to update the relative size scores or relative amount scores for the other elements contained in the input or set of inputs. As an example, this process may be done iteratively, such as an example where the apple is recognized and used to update the relative size scores or relative amount scores, and the fork is subsequently recognized and used to refine the relative size scores or relative amount scores of the input or set of inputs further.

Computer vision, as disclosed herein, may be used to generate one or more exchangeable representations of the disentangled information of an input or set of inputs. Said exchangeable representations of the disentangled information may take the form of computer readable media such as binary, ASCII, CSV, JSON, XML, and the like. Said exchangeable representations may further comprise a human readable element such as CSV, JSON, XML, YAML, and the like. Computer vision, as disclosed herein, may be used to generate one or more exchangeable representations of disentangled information from a FNF machine learning model, disclosed herein. Computer vision, as disclosed herein, may be used to generate one or more exchangeable representations of disentangled information from a FRM machine learning model, disclosed herein. Computer vision as disclosed herein may be used to generate one or more exchangeable representations of disentangled information from a combination of one or more FNF machine learning models disclosed herein and one or more FRM machine learning models disclosed herein.

The systems, the methods, the computer-readable media, and the techniques disclosed herein may comprise using computer vision to determine one or more Health Scores for an input or set of inputs. Determination of the one or more Health Scores may comprise using one or more FNF machine learning models, disclosed herein to determine the food-likeliness score of an input or set of inputs. This determination may further comprise using one or more FRM machine learning models, disclosed herein, to determine the presence and amount of a plurality of nutriments, such as those contained in one or more comprehensive nutriment databases in the input or set of inputs. This determination may further comprise using a plurality of nutritional information contained in one or more comprehensive nutriment databases, in combination with nutriment-likeliness and relative size and amount scores to determine one or more overall nutritional scores for an input or set of inputs. This determination may further comprise subsequent ML, CNN, DNN, RNN, LLM, and LFM operations as disclosed herein to further refine the one or more overall nutritional scores for an input or set of inputs. These one or more nutritional scores, as disclosed herein, may at least in part be utilized to determine an overall Health Score for an input or set of inputs.

Object detection is a technique used in computer vision for both (1) identification and (2) localization of objects within an image or a video. Image identification aims to predict the class of an image or an object within an image into one of a category or class. Examples of categories or classes may include, e.g., human, animal, car, tree, bird, shirt, ocean, happy, outdoors, etc. Image localization is the process of identifying the correct location of one or multiple objects. Bounding boxes may be used in object detection techniques and image localization within computer vision. Bounding boxes may be annotation markers drawn around objects in an image (e.g., a frame of a video). Bounding boxes may be often, although not always, rectangularly shaped. In some cases, bounding boxes may be applied by humans to training data sets. However, bounding boxes may also be applied to images by a trained machine learning that is trained to detect one or more different objects (e.g., humans, hands, faces, cars, etc.). In addition or in alternative to bounding boxes, detection and tracking techniques may use any object detection annotation techniques, such as semantic segmentation, instance segmentation, polygon annotation, non-polygon annotation, landmarking, 3D cuboids, etc.

### Neural Networks

The systems, the methods, the computer-readable media, and the techniques disclosed herein may implement one or more convolutional neural networks (CNN). Neural networks are a subset of machine learning and are often at the core of many deep learning algorithms. Neural networks may comprise node layers, each which may comprise one or more of an input layer, one or more hidden layers, and an output layer. Each node of a neural network may connect to another node of the neural network. Each node of a neural network may have an associated weight and threshold. In some cases, if an output from any individual node of a neural network is above a specified threshold value, that node is activated, thereby sending data to the next layer of the neural network; otherwise, no data is passed along to the next layer of the neural network.

CNNs are a type of neural network. CNNs are often used for classification and computer vision tasks. Prior to CNNs, manual, time-consuming feature extraction methods were used to identify objects in images. However, CNNs provide a more scalable approach to image classification and object recognition tasks, leveraging principles from linear algebra, specifically matrix multiplication, to identify patterns within an image. That said, CNNs can be computationally demanding, using graphical processing units (GPUs) to train models.

CNNs may be distinguished from other neural networks by their superior performance with image, speech, or audio signal inputs. CNNs may comprise three main types of layers: convolutional layers, pooling layers, and fully-connected (FC) layers. The convolutional layer may be the first layer of a CNN. While convolutional layers can be followed by additional convolutional layers or pooling layers, the fully-connected layer may be the final layer of the CNN.

When applied to computer vision tasks within images, with each layer, the CNN increases in its complexity, identifying greater portions of the image. Earlier layers of a CNN may focus on simple features of an image, such as colors and edges. As the image data progresses through the layers of the CNN, the CNN starts to recognize larger elements or shapes of objects in the image until the CNN identifies the intended object.

The convolutional layer is a core building block of a CNN and may be where much of the computation of the CNN occurs. Convolution layers may use components including input data, a filter, and a feature map. Provided, for example, the input data comprises a color image (which e.g., includes a matrix of pixels in 3D), the input may have three dimensions—a height, width, and depth—which correspond to RGB in an image. CNNs may further comprise a feature detector (also known as a kernel or a filter), which moves across receptive fields of the image, checking if a feature is present. This process may be known as a convolution.

The feature detector may include a filter that is a two-dimensional array of weights, which represents part of an image. Filters of feature detectors may vary in size (e.g., 3×3 matrix), and the size may determine the size of the receptive field. The filter may be applied to an area of the image, and a dot product may be calculated between input pixels and the filter. The dot product may then be fed into an output array. Afterwards, the filter may shift by a stride, repeating the process until the filter has swept across the entire image. The final output from the series of dot products from the input and the filter may be known as a feature map, activation map, or a convolved feature. After each convolution operation, a CNN may apply a Rectified Linear Unit (ReLU) transformation to the feature map, introducing nonlinearity to the CNN.

In some cases, another convolution layer can follow the initial convolution layer of the CNN. For example, the structure of the CNN can become hierarchical as the later layers can see the pixels within the receptive fields of prior layers. As an example, assume a CNN used to determine if an image contains a bicycle. Each individual part of the bicycle (e.g., frame, handlebars, wheels, pedals, etc.) makes up a lower-level pattern in the CNN, and the combination of the parts represents a higher-level pattern, creating a feature hierarchy within the CNN.

The pooling layers, also known as downsampling, are further layers of a CNN. Pooling layers may conduct dimensionality reduction, reducing the number of parameters in the input (e.g., image, video, audio, etc.). Similar to the convolutional layer, the pooling layer sweeps a filter across the entire input, but, unlike the convolution layers, the filters of the pooling layers do not have any weights. Instead, the filters of the pooling layers apply an aggregation function to values within the receptive field, populating the output array. There are two main types of pooling: max pooling and average pooling. Max pooling may comprise moving the filter across the input to select the pixel with the maximum value to send to the output array. Average pooling may comprise moving the filter across the input to calculate the average value within the receptive field to send to the output array. While a lot of information is lost in the pooling layer, the pooling layer also has a number of benefits to the CNN. For example, pooling layers may help to reduce complexity of the CNN, improve efficiency, and limit risk of overfitting of the CNN.

Fully-connected layers are the final layer of a CNN. As previously disclosed, pixel values of an input image are not directly connected to output layers in partially connected layers. However, in the fully-connected layer, each node in the output layer connects directly to a node in the previous layer. The FC layer performs the task of classification based on the features extracted through the previous layers and their different filters. While convolutional layers and pooling layers tend to use ReLu functions, FC layers may leverage a softmax activation function to classify inputs appropriately, producing a probability from 0 to 1.

Neural networks, such as CNNs disclosed herein, may be employed in the systems, the methods, the computer-readable media, and the techniques disclosed herein. Neural networks, such as CNNs disclosed herein, may be utilized in the food recognition operation of the FNF machine learning model disclosed herein. Neural networks, such as CNNs disclosed herein, may be utilized in concert with disentangled information contained in images and videos input into the FNF model, as disclosed herein. Neural networks, such as CNNs describe herein, may be utilized in concert with the disentangled information to determine a food-likeliness score. This process may involve recursive operations, as disclosed herein, to refine the food-likeliness score of the input or set of inputs.

Neural networks, such as CNNs disclosed herein, may be utilized in the identification of nutriment items of the FRM machine learning model disclosed herein. Neural networks, such as CNNs disclosed herein, may be utilized in concert with the disentangled symbolic information contained in images and videos input into the FRM model, as disclosed herein. Neural networks, such as CNNs disclosed herein, may be utilized in concert with said disentangled information to determine a nutriment-likeliness score for a plurality of nutriments contained in one or more comprehensive nutriment databases. This process may involve recursive operations, as disclosed herein, to refine the nutriment-likeliness score of the input or set of inputs.

Neural networks, such as CNNs disclosed herein, may be utilized in the identification of relative sizes and amounts of the FRM machine learning model disclosed herein. Neural networks, such as CNNs disclosed herein, may be utilized in concert with the disentangled symbolic information contained in images and videos input into the FRM model, as disclosed herein. Neural networks, such as CNNs disclosed herein, may be utilized in concert with said disentangled information to determine one or more relative size scores or relative amount scores for an element of an input or set of inputs. Neural networks, such as CNNs disclosed herein, may be utilized to identify nutriment elements in an input or set of inputs. Neural networks, such as CNNs disclosed herein, may be utilized to identify non-nutriment elements in an input or set of inputs. Neural networks, such as CNNs disclosed herein, may be utilized in concert with historical information to update relative size scores or relative amount scores for an element of an input or set of inputs. Neural networks, such as CNNs disclosed herein, may be utilized to refine the relative size scores or relative amount scores recursively, as disclosed herein. Neural networks, such as CNNs disclosed herein, may be used to generate one or more exchangeable representations of the disentangled information of an input or set of inputs. Said exchangeable representations of the disentangled information may take the form of computer readable media such as binary, ASCII, CSV, JSON, XML, and the like. Said exchangeable representations may further comprise a human readable element such as CSV, JSON, XML, YAML, and the like. Neural networks, such as CNNs disclosed herein, may be used to generate one or more exchangeable representations of disentangled information from a FNF machine learning model, disclosed herein. Neural networks, such as CNNs disclosed herein, may be used to generate one or more exchangeable representations of disentangled information from a FRM machine learning model, disclosed herein. Neural networks, such as CNNs disclosed herein, may be used to generate one or more exchangeable representations of disentangled information from a combination of one or more FNF machine learning models disclosed herein and one or more FRM machine learning models disclosed herein.

### Language Models

The systems, the methods, the computer-readable media, and the techniques disclosed herein may implement a language model. At a high level, a language model is a probabilistic model of a natural language that can generate probabilities of a series of words, based on text corpora in one or multiple languages it was trained on. Example applications of language models may include speech recognition, machine translation, natural language generation, optical character recognition, handwriting recognition, grammar induction, information retrieval, malware analysis, content creation, search structuring, machine assistance, customer support, cyber defense, code development, transcription generation, market research, keyword research, sales automation, sentiment analysis, risk analysis, copywriting, autocompletion, shell command generation, regex generation, SQL generation, code review, database optimization, DevOps automation, frontend or website generation, product documentation generation, personalized tutoring, decision making, etc.

In some cases, language models may implement statistical or probabilistic techniques. Statistical language models may use statistical patterns in data to make predictions about the likelihood of specific sequences of words. For example, one technique for building a probabilistic language model is to calculate n-gram probabilities (e.g., a sequence of words, where n is a number greater than zero). In some cases, a statistical language model may calculate likelihood of different n-grams (word combinations) in a text. One example technique for calculating likelihood may include counting the number of times each word combination appears to obtain a first value. Then, the first value may be divided by the number of times the previous word appears. This technique is based on Markov assumption (e.g., probability of a word combination (the future) depends only on the previous word (the present) and not the words that came before it (the past)). Types of n-gram models may include unigrams (e.g., evaluate each word independently), bigrams (e.g., consider probability of a word given the previous word), trigrams (e.g., consider probability of a word given the previous two words), etc. While n-grams may be efficient, n-gram language models may be challenged in considering long-term context of words in a sequence. Other statistical language models may include, for example, exponential language models (e.g., maximum entropy language models), skip-gram language models, etc.

In some cases, language models may implement machine learning techniques. For example, language models may be neural language models. Advantageously, neural language models may be able to capture context better than statistical models. Also, neural language models may handle more complex language structures and longer dependencies between words than statistical language models.

In some cases, neural language models may implement deep neural networks (DNN). DNN language models use DNNs to predict the likelihood of a sequence of words. DNN language models may be trained on a large corpus of text data and are capable of learning the underlying structure of the language. DNN language models can handle large vocabularies and deal with rare or unknown words by using distributed representations. One simple example of a DNN language model may comprise an input layer, a hidden layer, another hidden layer, and an output layer.

In some cases, DNN language models may use recurrent neural networks (RNNs) or transformer networks. A RNN language model may “remember” previous outputs when receiving next inputs. This is in contrast to other neural networks in which inputs and outputs are independent of each other. RNN language models are particularly useful in predicting the next word in a sentence, as RNN language models consider previous words in an input (e.g., sentence). One feature of RNN language models is a hidden state vector that remembers information about a sequence. This “memory” allows RNN language models to remember information that has been previously calculated and use this information to make future predictions. The hidden state is maintained by a hidden layer in the RNN language model.

In some cases, RNN language models can be computationally expensive for very long input sequences. This means that the RNN language model's ability to make accurate predictions based on the information from the initial words of the sentence decreases. In some cases, this the “vanishing gradients” problem may be addressed using, for example, a Long Short-term Memory (LSTM) architecture that may be the same as or similar to the LSTM disclosed herein. In some cases, RNN language models generate continuous representations or embeddings of words. Such continuous space embeddings may help to alleviate the “curse of dimensionality,” which is the consequence of the number of possible sequences of words increasing exponentially with the size of the vocabulary, furtherly causing a data sparsity problem. In some cases, RNN language models reduce this challenge by representing words as non-linear combinations of weights in a neural net.

A large language model (LLM) is a language model characterized by its large size. LLMs may be based on machine learning techniques. In some cases, LLMs may be implemented as data structures, programs, algorithms, architectures, or the like, that may be designed to interpret natural language. LLMs may generate text responses in response to text based prompts. LLMs may be neural networks trained on large collections of natural language source documents. Accordingly, in some cases, LLMs may be trained to generate predictive responses based on provided prompts. LLM prompts may include context information, examples, or the like, that may enable LLMs to generate responses directed to specific queries.

LLMs may be very big and may run on specialist hardware. LLMs may be expensive to train and run and use masses amounts of data. LLMs may accomplish such a large size via artificial intelligence accelerators, which are able to process vast amounts of text data, mostly scraped from the Internet. LLMs may use neural networks with, for example, millions, billions, trillions, etc. weights. In some cases, LLMs may be (pre-)trained using self-supervised learning and semi-supervised learning. In some cases, LLMs may be (pre-)trained using unsupervised learning. In some cases, LLMs may use a transformer architecture for faster training. In some cases, LLMs may use alternative architectures such as mixture of experts (MoE).

In some cases, LLMs obtain an input text and repeatedly predict the next token or word. In some cases, fine tuning may be used to adapt LLMs to accomplish specific tasks. Larger LLMs sized models may be prompt-engineered to achieve similar results. LLMs may acquire embodied knowledge about syntax, semantics, and “ontology” inherent in human language corpora, but also inaccuracies and biases present in the corpora.

In some cases, a LLM may embed a plurality of large foundational models (LFMs), including both closed-source LFMs and open-source LFMs. For example, LFMs may include Azure OpenAI (complete and chat APIs for GPT-3, GPT-3.5, and GPT-4, GPT-o3, GPT-o3 mini used in ChatGPT), OpenAI (complete and chat APIs for GPT-3, GPT-3.5 and GPT-4, GPT-o3, GPT-o3 mini), Google Vertex AI (e.g., PaLM, PaLM-2), Meta's LLaMa, as well as BLOOM, Ernie 3.0 Titan, and Anthropic's Claude 2. For example, open-source LFMs may include FLAN-T5, OpenAssistant, RoBERTa, MiniLM, MPNet, their later versions, modified versions, and the like. For example, LFMs may include modified or later versions of those listed above. For example, LFMs may include open-source or modified open-source versions of those listed above.

Language models, such as LLMs disclosed herein, may be employed in the systems, the methods, the computer-readable media, and the techniques disclosed herein. Language models, such as LLMs disclosed herein, may be utilized in the food recognition operation of the FNF machine learning model disclosed herein. Language models, such as LLMs disclosed herein, may be utilized in concert with disentangled information contained in images and videos input into the FNF model, as disclosed herein. Language models, such as LLMs disclosed herein, may be utilized in concert with said disentangled information to determine a food-likeliness score. This process may involve recursive operations, as disclosed herein, to refine the food-likeliness score of the input or set of inputs.

Language models, such as LLMs disclosed herein, may be utilized in the identification of nutriment items of the FRM machine learning model disclosed herein. Language models, such as LLMs disclosed herein, may be utilized in concert with the disentangled symbolic information contained in images and videos input into the FRM model, as disclosed herein. Language models, such as LLMs disclosed herein, may be utilized in concert with said disentangled information to determine a nutriment-likeliness score for a plurality of nutriments contained in one or more comprehensive nutriment databases. This process may involve recursive operations, as disclosed herein, to refine the nutriment-likeliness score of the input or set of inputs.

Language models, such as LLMs disclosed herein, may be utilized in the identification of relative sizes and amounts of the FRM machine learning model disclosed herein. Language models, such as LLMs disclosed herein, may be utilized in concert with the disentangled symbolic information contained in images and videos input into the FRM model, as disclosed herein. Language models, such as LLMs disclosed herein, may be utilized in concert with said disentangled information to determine one or more relative size scores or relative amount scores for an element of an input or set of inputs. Language models, such as LLMs disclosed herein, may be utilized to identify nutriment elements in an input or set of inputs. Language models, such as LLMs disclosed herein, may be utilized to identify non-nutriment elements in an input or set of inputs. Language models, such as LLMs disclosed herein, may be utilized in concert with historical information to update relative size scores or relative amount scores for an element of an input or set of inputs. Language models, such as LLMs disclosed herein, may be utilized to refine the relative size scores or relative amount scores recursively, as disclosed herein.

Language models, such as LLMs disclosed herein, may be used to generate one or more exchangeable representations of the disentangled information of an input or set of inputs. Said exchangeable representations of the disentangled information may take the form of computer readable media such as binary, ASCII, CSV, JSON, XML, and the like. Said exchangeable representations may further comprise a human readable element such as CSV, JSON, XML, YAML, and the like. Language models, such as LLMs disclosed herein, may be used to generate one or more exchangeable representations of disentangled information from a FNF machine learning model, disclosed herein. Language models, such as LLMs disclosed herein, may be used to generate one or more exchangeable representations of disentangled information from a FRM machine learning model, disclosed herein. Language models, such as LLMs disclosed herein, may be used to generate one or more exchangeable representations of disentangled information from a combination of one or more FNF machine learning models disclosed herein and one or more FRM machine learning models disclosed herein.

It will be readily understood the examples of Machine Learning Models, Neural Networks, and Language Models described herein are illustrative and not limitative in nature. It will further be appreciated updated versions and new equivalents of the Machine Learning Models, Neural Networks, and Language models described herein may be employed without departing from the systems, the methods, the computer-readable media, and the techniques described herein.

### Grouped Inputs

Context from multiple input types and models may be grouped using metadata from raw inputs and model outputs to refine the nutrient score and Health Score for a particular meal or set of meals. As an example, a grouped input may contain a picture of the menu and pictures of individual items of the meal, each with time stamps within an hour our two of each other. The grouped input may further comprise location information about where each picture was taken. In this example, computer vision may be used to identify the words on the menu, as well as nutriments in the images of the meal. A LLM may be used to identify the items on the menu, and their nutrient and Health Scores, while a CNN determines the nutrient and Health Scores. These may be input into an RNN, an additional LLM, LFM, CNN, DNN etc. or any combination therein, and the combined contextual information from the menu and food images individually, as well as the time and location stamps, may be used to determine the likelihood these separate images are part of the same meal. The model could then update the nutrient and Health Scores for the grouped input of the menu and food images.

### User Inputs

Context from user inputs may be used to further refine the nutrient score and Health score for a particular meal or set of meals. Context from user data, user preferences, and authorized monitoring of other user parameters such as location, activity, exercise, etc. may be utilized to further refine the nutrient score and Health score. In following with the example of grouped inputs above, the user may be prompted: “It looks like you had the Caeser Salad and Fettuccine Alfredo with Chicken last night for dinner. Is this correct?.” This user input may be used to confirm nutrient or Health Scores from the FNF and FRM model. As another example, a user may upload a photo of the menu from a restaurant, and include text along with the image, stating: “I had the Caeser Salad and Fettuccine Alfredo with Chicken.” This user input may be used to produce or refine nutrient or Health Scores from the FNF and FRM model.

### Post Processing

Exchangeable representations of disentangled information, as disclosed herein, may undergo one or more post-processing operations. These post processing operations may comprise one or more neural networks, such as CNNs, DNNs, RNNs, and the like, as disclosed herein. These post processing operations may comprise one or more language models such as LLMs, LFMs and the like, as disclosed herein. These post processing operations may comprise formatting the exchangeable representations in a human readable form. These post processing operations may comprise updating a human readable food log. These post processing operations may comprise soliciting user input. These post processing operations may comprise proving user feedback. These post processing operations may comprise cloud computing and cloud storage. These post processing operations may comprise periodic checks of historical data. These post processing operations may comprise comparing local classifications with historical data. These post processing operations may comprise comparing local classifications with the results of cloud computing tasks.

### Cloud Computing

One or more operations of the FNF and FRM models disclosed herein may be completed using cloud computing. Cloud computing describes a process wherein at least a portion of a task or tasks is completed remotely on one or more external devices or servers, such that the result of the remotely completed task or tasks may be communicated back and utilized locally. In some cases, cloud computing may be used to offload the burden of computing power, memory, storage, and the like to a remote device. One or more exchangeable representations disclosed herein of disentangled information disclosed herein may be exchanged via a protocol such as HTTP, HTTPS, SRTP, SCTP, IPsec, DTLS, TCP, UDP, OpenVPN, and the like to one or more external servers for cloud computing. One or more operations of the FNF machine learning model disclosed herein may be completed using cloud computing. One or more operations of the FRM machine learning model disclosed herein may be completed using cloud computing. One or more cloud computing operations disclosed herein may be used recursively as disclosed herein.

### Examples of Computing Systems

Referring to FIG. 5, a block diagram is shown depicting an example machine that includes a computer system 500 (e.g., a processing or computing system) within which a set of instructions can execute for causing a device to perform or execute any one or more of the aspects or methodologies for static code scheduling of the present disclosure. The components in FIG. 5 are examples and do not limit the scope of use or functionality of any hardware, software, embedded logic component, or a combination of two or more such components with particular implementations.

Computer system 500 may include one or more processors 501, a memory 503, and a storage 508 that communicate with each other, and with other components, via a bus 540. The bus 540 may also link a display 532, one or more input devices 533 (which may, for example, include a keypad, a keyboard, a mouse, a stylus, a smart device, smart glasses, smart appliances, AR headsets, MR headsets, etc.), one or more output devices 534, one or more storage devices 535, and various tangible storage media 536. All of these elements may interface directly or via one or more interfaces or adaptors to the bus 540. For instance, the various tangible storage media 536 can interface with the bus 540 via storage medium interface 526. Computer system 500 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 500 includes one or more processor(s) 507 (e.g., central processing units (CPUs), general purpose graphics processing units (GPGPUs), or quantum processing units (QPUs)) that carry out functions. Processor(s) 501 optionally contains a cache memory unit 502 for temporary local storage of instructions, data, or computer addresses. Processor(s) 501 are configured to assist in execution of computer readable instructions. Computer system 500 may provide functionality for the components depicted in FIG. 5 as a result of the processor(s) 501 executing non-transitory, processor-executable instructions embodied in one or more tangible computer-readable storage media, such as memory 503, storage 508, storage devices 535, or storage medium 536. The computer-readable media may store software that implements particular operations, and processor(s) 501 may execute the software. Memory 503 may read the software from one or more other computer-readable media (such as mass storage device(s) 535, 536) or from one or more other sources through a suitable interface, such as network interface 520. The software may cause processor(s) 501 to carry out one or more processes or one or more operations of one or more processes described or illustrated herein. Carrying out such processes or operations may include defining data structures stored in memory 503 and modifying the data structures as directed by the software.

The memory 503 may include various components (e.g., machine readable media) including, but not limited to, a random access memory component (e.g., RAM 504) (e.g., static RAM (SRAM), dynamic RAM (DRAM), ferroelectric random access memory (FRAM), phase-change random access memory (PRAM), etc.), a read-only memory component (e.g., ROM 505), and any combinations thereof. ROM 505 may act to communicate data and instructions unidirectionally to processor(s) 501, and RAM 504 may act to communicate data and instructions bidirectionally with processor(s) 501. ROM 505 and RAM 504 may include any suitable tangible computer-readable media described below. In one example, a basic input/output system 506 (BIOS), including basic routines that help to transfer information between elements within computer system 500, such as during start-up, may be stored in the memory 503.

Fixed storage 508 is connected bidirectionally to processor(s) 501, optionally through storage control unit 507. Fixed storage 508 provides additional data storage capacity and may also include any suitable tangible computer-readable media disclosed herein. Storage 508 may be used to store operating system 509, executable(s) 510, data 511, applications 512 (application programs), and the like. Storage 508 can also include an optical disk drive, a solid-state memory device (e.g., flash-based systems), or a combination of any of the above. Information in storage 508 may, in appropriate cases, be incorporated as virtual memory in memory 503.

In one example, storage device(s) 535 may be removably interfaced with computer system 500 (e.g., via an external port connector (not shown)) via a storage device interface 525. Particularly, storage device(s) 535 and an associated machine-readable medium may provide non-volatile or volatile storage of machine-readable instructions, data structures, program modules, or other data for the computer system 500. In one example, software may reside, completely or partially, within a machine-readable medium on storage device(s) 535. In another example, software may reside, completely or partially, within processor(s) 501.

Bus 540 connects a wide variety of subsystems. Herein, reference to a bus may encompass one or more digital signal lines serving a common function, where appropriate. Bus 540 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 500 may also include an input device 533. In one example, a user of computer system 500 may enter commands or other information into computer system 500 via input device(s) 533. Examples of an input device(s) 533 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), a smart device (e.g. smart glasses, smart appliances, AR headsets, MR headsets), social media (e.g. mobile and web-based applications for sharing photos, videos, and other media), and any combinations thereof. In some cases, the input device is a Kinect, Leap Motion, or the like. Input device(s) 533 may be interfaced to bus 540 via any of a variety of input interfaces 523 (e.g., input interface 523) including, but not limited to, serial, parallel, game port, USB, FIREWIRE, THUNDERBOLT, or any combination of the above.

In some cases, when computer system 500 is connected to network 530, computer system 500 may communicate with other devices, specifically mobile devices and enterprise systems, distributed computing systems, cloud storage systems, cloud computing systems, and the like, connected to network 530. Communications to and from computer system 500 may be sent through network interface 520. For example, network interface 520 may receive incoming communications (such as requests or responses from other devices) in the form of one or more packets (such as Internet Protocol (IP) packets) from network 530, and computer system 500 may store the incoming communications in memory 503 for processing. Computer system 500 may similarly store outgoing communications (such as requests or responses to other devices) in the form of one or more packets in memory 503 and communicated to network 530 from network interface 520. Processor(s) 501 may access these communication packets stored in memory 503 for processing.

Examples of the network interface 520 include, but are not limited to, a network interface card, a modem, and any combination thereof. Examples of a network 530 or network segment 530 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 530, may employ a wired or a wireless mode of communication. In general, any network topology may be used.

Information and data can be displayed through a display 532. Examples of a display 532 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 532 may be a mounted display, such as a Television or computer monitor, a mobile display, such as a laptop or smartphone, a wearable display, such as smart glasses, AR headset, MR headset, smart watch, and any combinations thereof. The display 532 can interface to the processor(s) 501, memory 503, and fixed storage 508, as well as other devices, such as input device(s) 533, via the bus 540. The display 532 is linked to the bus 540 via a video interface 522, and transport of data between the display 532 and the bus 540 can be controlled via the graphics control 521. In some cases, the display is a video projector. In some cases, the display is a head-mounted display (HMD) such as a VR headset. In further cases, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further cases, the display is a combination of devices such as those disclosed herein.

In addition to a display 532, computer system 500 may include one or more other peripheral output devices 534 including, but not limited to, an audio speaker, a printer, a storage device, and any combinations thereof. Such peripheral output devices may be connected to the bus 540 via an output interface 524. Examples of an output interface 524 include, but are not limited to, a serial port, a parallel connection, a USB port, a FIREWIRE port, a THUNDERBOLT port, and any combinations thereof.

In addition or as an alternative, computer system 500 may provide functionality as a result of logic hardwired or otherwise embodied in a circuit, which may operate in place of or together with software to execute one or more processes or one or more operations of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Various illustrative logical blocks, modules, circuits, and algorithm operations described in connection with the examples disclosed herein may be implemented as electronic hardware, computer software, or combinations of both. To clearly illustrate this interchangeability of hardware and software, various illustrative components, blocks, modules, circuits, and operations have been described above generally in terms of their functionality.

The various illustrative logical blocks, modules, and circuits described in connection with the examples disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions disclosed herein. A general purpose processor may be a microprocessor, but in the alternative, the processor may be any processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The operations of a method, a technique, or an algorithm described in connection with the examples disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An example storage medium may be coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Select televisions, video players, and digital music players with optional computer network connectivity may be suitable for use in the system disclosed herein. Suitable tablet computers, in various cases, include those with booklet, slate, and convertible configurations.

In some cases, the computing device includes an operating system configured to perform executable instructions. The operating system is, for example, software, including programs and data, which manages the device's hardware and provides services for execution of applications. Suitable server operating systems may include, by way of non-limiting examples, FreeBSD, OpenBSD, NetBSD®, Linux, Apple® Mac OS X Server®, Oracle® Solaris®, Windows Server®, and Novell® NetWare® Suitable personal computer operating systems may include, by way of non-limiting examples, Microsoft® Windows®, Apple® Mac OS X®, UNIX®, and UNIX-like operating systems such as GNU/Linux®. In some cases, the operating system is provided by cloud computing. Suitable mobile smartphone operating systems may include, by way of non-limiting examples, Nokia® Symbian® OS, Apple® iOS®, Research In Motion® BlackBerry OS®, Google® Android®, Microsoft® Windows Phone& OS, Microsoft® Windows Mobile® OS, Linux®, and Palm® WebOS®.

In some cases, the systems, the methods, the computer-readable media, and the techniques disclosed herein include one or more non-transitory computer readable storage media encoded with a program including instructions executable by the operating system of an optionally networked computing device. In further cases, a computer readable storage medium is a tangible component of a computing device. In still further cases, a computer readable storage medium is optionally removable from a computing device. In some cases, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

In some cases, the systems, the methods, the computer-readable media, and the techniques disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. A computer program may be written in various versions of various languages.

The functionality of the computer readable instructions may be combined or distributed in various ways across various environments. In some cases, a computer program comprises one sequence of instructions. In some cases, a computer program comprises a plurality of sequences of instructions. In some cases, a computer program is provided from one location. In some cases, a computer program is provided from a plurality of locations. In some cases, a computer program includes one or more software modules. In some cases, a computer program includes, in part or in whole, one or more web applications, one or more mobile applications, one or more standalone applications, one or more web browser plug-ins, extensions, add-ins, or add-ons, or combinations thereof.

In some cases, a computer program includes a web application. A web application, in various cases, may utilize one or more software frameworks and one or more database systems. In some cases, a web application is created upon a software framework such as Microsoft® .NET or Ruby on Rails (RoR). In some cases, a web application utilizes one or more database systems including, by way of non-limiting examples, relational, non-relational, object oriented, associative, XML, and document oriented database systems. In further cases, suitable relational database systems include, by way of non-limiting examples, Microsoft® SQL Server, mySQL™, and Oracle®. A web application, in some cases, may be written in one or more versions of one or more languages. A web application may be written in one or more markup languages, presentation definition languages, client-side scripting languages, server-side coding languages, database query languages, or combinations thereof. In some cases, a web application is written to some extent in a markup language such as Hypertext Markup Language (HTML), Extensible Hypertext Markup Language (XHTML), or extensible Markup Language (XML). In some cases, a web application is written to some extent in a presentation definition language such as Cascading Style Sheets (CSS). In some cases, a web application is written to some extent in a client-side scripting language such as Asynchronous Javascript and XML (AJAX), Flash® ActionScript, JavaScript, or Silverlight®. In some cases, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some cases, a web application is written to some extent in a database query language such as Structured Query Language (SQL). In some cases, a web application integrates enterprise server products such as IBM® Lotus Domino®. In some cases, a web application includes a media player element. In some cases, a media player element utilizes one or more of many suitable multimedia technologies including, by way of non-limiting examples, Adobe® Flash®, HTML 5, Apple® QuickTime®, Microsoft® Silverlight®, Java™, and Unity®.

In some cases, a computer program includes a mobile application provided to a mobile computing device. In some cases, the mobile application is provided to a mobile computing device at the time it is manufactured. In other cases, the mobile application is provided to a mobile computing device via the computer network disclosed herein.

In view of the disclosure provided herein, a mobile application may be created using hardware, languages, and development environments known to the art. In some cases, mobile applications are written in several languages. Suitable programming languages may include, by way of non-limiting examples, C, C++, C#, Objective-C, Java™, JavaScript, Pascal, Object Pascal, Python™, Julia, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums may be available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, and Samsung® Apps.

In some cases, a computer program includes a standalone application, which is a program that is run as an independent computer process, not an add-on to an existing process, e.g., not a plug-in. Standalone applications may be compiled. A compiler may be a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Julia, Visual Basic, and VB .NET, or combinations thereof. Compilation is often performed, at least in part, to create an executable program. In some cases, a computer program includes one or more executable complied applications.

In some cases, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Web browser plug-ins may include Adobe Flash® Player, Microsoft® Silverlight®, and Apple® QuickTime®. In some cases, the toolbar comprises one or more web browser extensions, add-ins, or add-ons. In some cases, the toolbar comprises one or more explorer bars, tool bands, or desk bands.

Several plug-in frameworks may be available that enable development of plug-ins in various programming languages, including, by way of non-limiting examples, C++, Delphi, Java™, PHP, Python™, Julia, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla Firefox®, Google® Chrome, Apple® Safari®, Opera Software® Opera®, and KDE Konqueror. In some cases, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

In some cases, the systems, the methods, the computer-readable media, and the techniques disclosed herein include software, server, or database modules, or use of the same. Software modules may be created by techniques using machines, software, and languages. The software modules disclosed herein are implemented in a multitude of ways. In some cases, a software module comprises a file, a section of code, a programming object, a programming structure, a distributed computing resource, a cloud computing resource, or combinations thereof. In some cases, a software module comprises a plurality of files, a plurality of sections of code, a plurality of programming objects, a plurality of programming structures, a plurality of distributed computing resources, a plurality of cloud computing resources, or combinations thereof. In some cases, the one or more software modules comprise, by way of non-limiting examples, a web application, a mobile application, a standalone application, and a distributed or cloud computing application. In some cases, software modules are in one computer program or application. In some cases, software modules are in more than one computer program or application. In some cases, software modules are hosted on one machine. In some cases, software modules are hosted on more than one machine. In some cases, software modules are hosted on a distributed computing platform such as a cloud computing platform. In some cases, software modules are hosted on one or more machines in one location. In some cases, software modules are hosted on one or more machines in more than one location.

In some cases, the systems, the methods, the computer-readable media, and the techniques disclosed herein include one or more databases, or use of the same. In some cases, various databases may be suitable for storage and retrieval of one or more of (i) wearable data, (ii) responses to health queries, (iii) geographic data, etc., one or more of which may be historical, present, or future data or information. In some cases, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some cases, a database is Internet-based. In further cases, a database is web-based. In still further cases, a database is cloud computing-based. In a particular case, a database is a distributed database. In other cases, a database is based on one or more local computer storage devices.

### Certain Definitions and Additional Considerations

Unless otherwise defined, all technical terms used herein have the same meaning as commonly understood by one of ordinary skill in the art to which this invention belongs.

As used in this specification and the appended claims, the terms “machine learning,” “machine learning techniques,” “machine learning algorithm,” “machine learning operation,” and “machine learning model” generally refer to any system or analytical or statistical procedure that may progressively improve computer performance of a task.

As used in this specification and the appended claims, the singular forms “a,” “an,” and “the” include plural references unless the context clearly dictates otherwise.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out.

The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

As used herein, “or” is intended to mean an “inclusive or” or what is also known as a “logical OR,” wherein when used as a logic statement, the expression “A or B” is true if either A or B is true, or if both A and B are true, and when used as a list of elements, the expression “A, B or C” is intended to include all combinations of the elements recited in the expression, for example, any of the elements selected from the group consisting of A, B, C, (A, B), (A, C), (B, C), and (A, B, C); and so on if additional elements are listed. As such, any reference to “or” herein is intended to encompass “and/or” unless otherwise stated.

It will be understood that when an element such as a layer, region, or substrate is referred to as being “on” or extending “onto” another element, it may be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it may be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it may be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

It will be understood that, although the terms “first,” “second,” “third,” etc. may be used herein to describe various elements, these elements should not be limited by these terms. These terms are only used to distinguish one element from another. For example, a first element may be termed a second element, and, similarly, a second element may be termed a first element, without departing from the scope of the present disclosure.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, like characters refer to like elements.

While preferred embodiments of the present invention have been shown and disclosed herein, it will be obvious to those skilled in the art that such embodiments are provided by way of example only. Numerous variations, changes, and substitutions will now occur to those skilled in the art without departing from the invention. It should be understood that various alternatives to the embodiments of the invention disclosed herein may be employed in practicing the invention. It is intended that the following claims define the scope of the invention and that methods and structures within the scope of these claims and their equivalents be covered thereby.

It should be noted that various illustrative or suggested ranges set forth herein are specific to their example embodiments and are not intended to limit the scope or range of disclosed technologies, but, again, merely provide example ranges for frequency, amplitudes, etc. associated with their respective embodiments or use cases. Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

It should be understood that, unless a term is expressly defined in this patent, there is no intent to limit the meaning of that term, either expressly or by implication, beyond its plain or ordinary meaning, and such term should not be interpreted to be limited in scope based at least in part on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are disclosed herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as disclosed herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, hardware modules may encompass a tangible entity, be that an entity that is physically constructed, permanently configured (e.g., hardwired), or temporarily configured (e.g., programmed) to operate in a certain manner or to perform certain operations disclosed herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and may operate on a resource (e.g., a collection of information). Elements that are described as being coupled and or connected may refer to two or more elements that may be (e.g., direct physical contact) or may not be (e.g., electrically connected, communicatively coupled, etc.) in direct contact with each other, but yet still cooperate or interact with each other.

The various operations of example methods disclosed herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines disclosed herein may be at least partially processor-implemented. For example, at least some of the operations of a method may be performed by one or more processors or processor-implemented hardware modules. The performance of certain operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain operations may be distributed among the one or more processors, not only residing within a single machine, but deployed across a number of machines. In some example embodiments, the one or more processors or processor-implemented modules may be located in a single geographic location (e.g., within an office environment, or a server farm). In other example embodiments, the one or more processors or processor-implemented modules may be distributed across a number of geographic locations.

### EXAMPLES

The examples and embodiments disclosed herein are for illustrative purposes only and various modifications or changes suggested to persons skilled in the art are to be included within the spirit and purview of this application and scope of the appended claims.

### Example 1: Background Tracking

Provided herein is an example of background tracking as described and claimed. A user may download an application of the claimed invention, and be prompted to complete several operations in an initialization process. One such operation of the initialization process may comprise granting user permission for background tracking. This user permission may comprise a request to access certain information on the device such as cameral roll information, real-time location, calendar events, and the like (e.g., for a specified duration of time such as, “all camera roll images in the past week,” “location information when using the application,” or “calendar events and invites which mention meals,” etc.). This background tracking may comprise gathering context from metadata, image, and video information contained in the camera roll of the device. This context may be used to generate trends at the time of initialization, in contrast to requiring a body of new inputs to begin generating trends and advice. As an example, a user may grant permission to access image information only. The images in the camera roll can be fed through the FNF or the FRM disclosed herein to generate historical data points for the types and quantities of food the user has taken photos of in the past. As another example, the user may grant permission to access photos as well as date and location stamps. If six out of seven meals photographed in the last week have location stamps at restaurants, this provides context for food trends as new images are identified. This trend information may be updated at a regular interval, which may be automatic or user specified.

### Example 2: Food-No-Food Modeling

Provided herein is an example of a Food-No-Food (FNF) machine learning model as described and claimed. The FNF model may be a lightweight (computationally inexpensive) model designed to run locally on the device where the application of the invention is installed. This FNF model may use computer vision and one or more neural networks and language models as disclosed herein to generate disentangled information from an input or set of inputs. This disentangled information may be processed locally using one or more neural networks and language models as disclosed herein to determine a food-likeliness score. The food-likeliness score may be determine recursively as disclosed herein. Inputs or sets of inputs with high food-likeliness scores (e.g., satisfying a threshold) may be passed for external processing by cloud computing as disclosed herein. Exchangeable representations of disentangled information from an input or set of inputs with a high food-likeliness score may be passed for external processing by cloud computing as disclosed herein. The external processing by cloud computing may comprise one or more FNF operations as disclosed herein. The external processing by cloud computing may comprise one or more FRM operations as disclosed herein. The external processing by cloud computing may comprise conversion of the exchangeable representations of disentangled information into human readable format as disclosed herein. The external processing by cloud computing may comprise conversion of the output of the one or more FNF operations into human readable form as disclosed herein. The external processing by cloud computing may comprise conversion of the output of the one or more FRM operations into human readable form as disclosed herein. The external processing by cloud computing may comprise communicating the results of one or more remote FNF operations back to the local device. The external processing by cloud computing may comprise communicating the results of one or more FRM operations back to the local device. The external processing by cloud computing may comprise communicating the results of one or more FNF and one or more FRM operations back to the local device.

### Example 3: Health, Fitness, and Wellness Goals

Provided herein is an example of goal tracking as claimed and described. Goal tracking may include health tracking, fitness tracking, wellness goals, exercise goals, weight loss goals, nutritional goals, and the like. The invention as claimed and described may comprise using context from one or more goal tracking inputs from the user to provide user suggestions. These user inputs may comprise food intake suggestions, restaurant recommendations, menu advice, and the like. As an example, if a user has expressed nutrient goals to reduce sodium intake, goal tracking may comprise an application alert stating: “Your estimated sodium intake today is 3,000 mg. Consider a low sodium option.” As an example, if a user has expressed weight loss goals, goal tracking using location information and user background on the time of day they eat meals could comprise an application alert stating: “Three restaurants in your area mention low-calorie options on their menu.” As an example, if a use is using the app to track nutritional goals in combination with daily exercise, goal tracking using activity tracking from a smart watch or other activity tracking device could comprise an application alert stating: “It looks like you've burned X calories today. Consider high protein meal options for dinner.” As an example, the application may connect with smart appliances to control the smart appliances for preparing, presenting, or discarding food. For example, if the application recommends eating a frozen chicken, the application may provide a control signal to a freezer to begin defrosting a chicken and a control signal to an over to pre-heat. As an example, the application may connect with food delivery services to cause delivery of a food or navigate a food delivery vehicle to a certain locations. For example, if the application recommends eating a salad, the application may transmit a control signal to a food delivery service to deliver a salad to a user's door, via, for example, an autonomous food delivery vehicle (e.g., an unmanned aerial vehicle, a self-driving car, an autonomous food delivery cart, etc.). These examples are not meant to be exhaustive, and it will be appreciated they may be used in any number and combination as tailored by the user's individual requirements.

### Example 4: Managing or Treating an Illness

Provided herein is an example of management or treatment of an illness, allergy, sensitivity, or other condition as claimed and described. Management or treatment of an illness, allergy, sensitivity, eating disorders (e.g., anorexia, anorexia, nervosa, bulimia, binge-eating, etc.), or other condition as claimed and disclosed herein may comprise one or more of allergen alerts, irritant alerts, restaurant recommendations, menu advice, food intake suggestions, and the like. As an example, if a user has celiac disease, management or treatment of an illness may comprise an alert such as: “Three restaurants in your area mention celiac-friendly options on their menu.” As an example, if a user has a dairy allergy, management or treatment of an illness in combination with the camera roll may comprise an alert such as: “Seven dairy-free options were found on the menu you just took a photo of.” These examples are not meant to be exhaustive, and it will be appreciated they may be used in any number of combinations as tailored by the user's individual requirements.

1.-37. (canceled)

38. A mobile device, comprising:
a camera;
a display;
one or more processors; and
one or more memories storing computer-executable instructions that, when executed, cause the one or more processors to perform operations comprising:
(A) receiving permission to access a camera roll stored in a first software application stored on said one or more memories, wherein said camera roll comprises a plurality of images that span at least one week, and wherein at least some of said plurality of images were captured with said camera;
(B) accessing said plurality of images in a second software application stored on said one or more memories;
(C) using a first machine learning model that is stored in said second software application, identifying a subset of said plurality of images that comprise a nutriment;
(D) providing said subset of said plurality of images as input to a second machine learning model;
(E) obtaining a prediction, from said second machine learning model, comprising a plurality of nutriment parameters corresponding to each nutriment of each image of said plurality of images; and
(F) presenting, using said display, an insight to a user on a graphical user interface of said second software application, wherein said insight is determined based at least in part on said plurality of nutriment parameters.

39. The mobile device of claim 38, wherein a different device from said mobile device captured at least one of said plurality of images.

40. The mobile device of claim 38, wherein said first machine learning model comprises a neural network and said second machine learning model comprises a large language model.

41. The mobile device of claim 38, wherein providing said subset of said plurality of images to said second machine learning model at (D) comprises transmitting said subset of said plurality of images to an external computing system, wherein said external computing system comprises said second machine learning model.

42. The mobile device of claim 38, wherein said plurality of nutriment parameters comprise two or more of: a type of nutriment, a quantity of nutriment, a size of nutriment, calories in nutriment, protein in nutriment, fats in nutriment, carbs in nutriment, or fiber in nutriment.

43. The mobile device of claim 38, wherein said insight comprises one or more of: a timeline of nutriment consumption, a trend or recommendation in consumption of one or more ingredients, a trend or recommendation in cooking preparation of one or more nutriments, a trend or recommendation in quantity of nutriment consumption, or a recommendation to align consumption with one or more goals of said user.

44. The mobile device of claim 38, further comprising:
(G) obtaining an indication that said user suffers from at least one medical condition, and wherein said insight comprises one or both of (i) recommending a nutriment for consumption based at least in part on said at least one medical condition or (ii) recommending a nutriment for avoidance based at least in part on said at least one medical condition.

45. The mobile device of claim 38, further comprising:
(G) capturing an additional image of an additional nutriment in response to an input from said user;
(H) obtaining an updated prediction, from said second machine learning model, comprising an additional plurality of nutriment parameters corresponding to said additional nutriment of said additional image; and
(I) presenting, using said display, an updated insight to said user on said graphical user interface, wherein said updated insight is determined based at least in part on said additional plurality of nutriment parameters.

46. The mobile device of claim 38, further comprising:
(G) presenting, using said display, said plurality of nutriment parameters to said user;
(H) obtaining, using said graphical user interface, feedback from said user; and
(I) revising said plurality of nutriment parameters based at least in part on said feedback.

47. The mobile device of claim 38, wherein said nutriment comprises one or more of: food, beverage, medication, supplement, vitamin, inhalant, or injection.

48. An imaging analysis method, comprising:
(A) obtaining permission to access a camera roll comprising a plurality of images that span at least one week, wherein said camera roll is accessible to a mobile device;
(B) at least in part in response to obtaining permission to access said image database at (A), analyzing each image of said plurality of images using a first machine learning model;
(C) obtaining, based at least in part on said analyzing in (B) a plurality of nutriment parameters, wherein each nutriment parameter of said plurality of nutriment parameters corresponds to each nutriment of each image of said plurality of images; and
(D) presenting an insight to said user on said mobile device, wherein said insight is determined based at least in part on said plurality of nutriment parameters.

49. The method of claim 48, wherein said plurality of images span at least one month.

50. The method of claim 48, wherein said mobile device comprises one or more of: a smartphone, a tablet, a smartwatch, or smart glasses.

51. The method of claim 48, wherein a camera of said mobile device captured at least a first subset of said plurality of images.

52. The method of claim 48, wherein a different device from said mobile device captured at least a second subset of said plurality of images.

53. The method of claim 48, wherein said camera roll operates within a first software application of said mobile device and operations (A)-(D) are performed within a second software application of said mobile device.

54. The method of claim 48, wherein said camera roll comprises one or more images posted to a social media accessible by said mobile device.

55. The method of claim 48, wherein said first machine learning model comprises a large language model.

56. The method of claim 48, wherein said plurality of nutriment parameters comprise two or more of: a type of nutriment, a quantity of nutriment, a size of nutriment, calories in nutriment, protein in nutriment, fats in nutriment, carbs in nutriment, or fiber in nutriment.

57. The method of claim 48, wherein said insight comprises one or more of: a timeline of nutriment consumption, a trend or recommendation in consumption of one or more ingredients, a trend or recommendation in cooking preparation of one or more nutriments, a trend or recommendation in quantity of nutriment consumption, or a recommendation to align consumption with one or more goals of said user.

58. The method of claim 48, further comprising:
(E) obtaining an indication that said user suffers from at least one medical condition, and wherein said insight comprises one or both of (i) recommending a nutriment for consumption based at least in part on said at least one medical condition or (ii) recommending a nutriment for avoidance based at least in part on said at least one medical condition.

59. The method of claim 48, further comprising:
(E) identifying said plurality of images from said camera roll using a second machine learning model configured to identify whether an image of said plurality of images comprises a nutriment.

60. The method of claim 48, further comprising:
(E) capturing an additional image of an additional nutriment in response to an input from said user;
(F) obtaining an updated prediction, from said first machine learning model, comprising an additional plurality of nutriment parameters corresponding to said additional nutriment of said additional image; and
(G) presenting an updated insight to said user on said mobile device, wherein said updated insight is determined based at least in part on said additional plurality of nutriment parameters.

61. The method of claim 48, wherein said nutriment comprises one or more of: food, beverage, medication, supplement, vitamin, inhalant, or injection.

62. The method of claim 48, wherein said obtaining comprises storing or evaluating one or more device-level permission parameters at said mobile device.
